(19) United States
(12) Patent Application Publication
(10) Pub. No.: US 2008/0167326 A1
(43) Pub. Date: Jul. 10, 2008

(54) Method For Assessing the Predisposition and/or Susceptibility to Copd by Analysing Fgf-Bp1

(75) Inventors: Witte Koopmann, Lund (SE); Simon Smith, Cheshire (GB)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 11/910,092

(22) PCT Filed: Apr. 3, 2006
(86) PCT No.: PCT/SE2006/000400

§ 371 Date: Oct. 3, 2007

(30) Foreign Application Priority Data
Apr. 7, 2005 (SE) 0500781-0

(51) Int. Cl. A61K 31/522; C12Q 1/68; G01N 33/53

(52) U.S. Cl. 514/263.32; 435/6; 435/7.1

(57) ABSTRACT

The present invention relates to the discovery of an association between the gene encoding FGF-BP1 (fibroblast growth factor-binding protein) and chronic obstructive pulmonary disease (COPD). The invention identifies a role for FGF-BP1 in COPD. The present invention therefore relates to diagnostic techniques for the detection of COPD by detecting all or part of this gene, its precursors or products (mRNA, cDNA, genomic DNA, or protein). The present invention also provides methods and as-says for identifying compounds which modulate FGF-BP1 and which may be used for treating respiratory diseases such as COPD. Furthermore, the invention relates to polymorphisms in the genes encoding FGF-BP1. The invention also relates to the use of polymorphisms in the FGF-BP1-encoding genes in assessing predisposition and/or susceptibility of an individual to chronic obstructive pulmonary disease (COPD).

### FIELD OF THE INVENTION

The present invention relates to the discovery of an association between the gene encoding FGF-BP1 (fibroblast growth factor-binding protein) and chronic obstructive pulmonary disease (COPD). Thus, the present invention identifies a role for FGF-BP1 in COPD. The present invention therefore relates to diagnostic techniques for the detection of COPD by detecting all or part of this gene, its precursors or products (mRNA, cDNA, genomic DNA, or protein). The present invention also provides methods and assays for identifying compounds which modulate FGF-BP1 and which may be used for treating respiratory diseases such as COPD. Furthermore, the invention relates to polymorphisms in the genes encoding FGF-BP1. The invention also relates to the use of polymorphisms in the FGF-BP1-encoding genes in assessing predisposition and/or susceptibility of an individual to chronic obstructive pulmonary disease (COPD).

### BACKGROUND TO INVENTION

The essential function of the lungs requires a fragile structure with enormous exposure to the environment, including pollutants, microbes, allergens, and carcinogens. Host factors, resulting from interactions of lifestyle choices and genetic composition, influence the response to this exposure. Damage or infection to the lungs can give rise to a wide range of diseases of the respiratory system (or respiratory diseases). A number of these diseases are of great public health importance. Respiratory diseases include Acute Lung Injury, Acute Respiratory Distress Syndrome (ARDS), occupational lung disease, lung cancer, tuberculosis, fibrosis, pneumoconiosis, pneumonia, emphysema, Chronic Obstructive Pulmonary Disease (COPD) and asthma.

The pathophysiology of COPD is complex and poorly understood. Current clinical guidelines define COPD as a disease state characterized by airflow limitation that is not fully reversible. The airflow limitation is usually both progressive and associated with an abnormal inflammatory response of the lungs to noxious particles and gases. The most important contributory source of such particles and gases, at least in the western world, is tobacco smoke. COPD patients have a variety of symptoms, including cough, shortness of breath, and excessive production of sputum; such symptoms arise from dysfunction of a number of cellular compartments, including neutrophils, macrophages, and epithelial cells.

Mucus hypersecretion, thickening of the mucus and impaired mucociliary clearance (MCC) are well-established features of COPD pathophysiology, and contribute significantly to the morbidity and mortality of the disease. Small airways can become occluded by mucus plugs, leading to severe airway obstruction. Additionally, excessive mucus production in large airways can result in symptoms of chronic bronchitis. Impaired MCC also gives rise to increased rates and severity of exacerbations.

There is a pressing need for the identification of genes that are involved in respiratory diseases such as COPD and which represent targets for therapy. This may enable the development of novel therapies for COPD by screening compounds and other entities, such as antibodies, which modulate the activity of the protein encoded by the associated genes. Knowledge of the sequence of the associated genes may also enable the development of novel gene therapy techniques, or RNAi-based techniques to treat COPD. The discovery of associated genes may also assist in developing novel methods for diagnosing COPD via (i) analysis of the pattern of genotypes of associated polymorphisms such as single nucleotide polymorphisms (SNPs), (ii) measuring the levels of transcribed mRNA present in affected tissue and (iii) measuring the levels of the protein in affected tissue. It is possible that the diagnosis of COPD, or the prediction of the predisposition to COPD, by these methods may be achieved in patients who do not yet display the classical symptoms of the disease. Such determination of susceptibility to COPD or the early detection of disease development may lead to earlier clinical intervention than is currently possible and may lead to more effective treatment of the disease.

FGF-BP1 (fibroblast growth factor-binding protein) has been allocated Genbank accession number AF149412. FGF-BP1 (also known as HBP17, for heparin-binding growth factor-binding protein of 17 kDa, and also HBp17, FGFBP1, and FGFBP) was first purified as a protein secreted from an epidermal carcinoma cell line (A431-AJC) (Wu et al., JBC 266:16778, 1991). The authors showed that purified FGF-BP1 bound noncovalently to both heparin-binding growth factor 1 and 2, and went on to demonstrate that the biological activity of both HBGF1 (also known as FGF1) and HBGF2 (also known as FGF2) was inhibited by FGF-BP1 binding. The corresponding cDNA was cloned and sequenced, and Northern analysis of a panel of cell lines showed high expression levels in a number of squamous cell carcinoma lines. FGF-BP1 has been shown by immunohistochemical analysis to be expressed at low levels in normal human skin and upregulated in squamous cell carcinomas (Mongiat et al., JBC 276:10263, 2001).

SEQ ID NO: 1 contains the genomic sequence of human FGF-BP1. This includes a 5′ non-transcribed region (spanning bases 1 to 7314), exon 1 (spanning residues 7315 to 7367), exon 2 (spanning residues 7583-7803), exon 3 (spanning residues 9402 to 10485) and a 3′ non-transcribed region (spanning residues 10486-10605). Note that 3 exons are predicted based upon examination of 13 distinct transcripts, which suggest that the coding exon (exon 3) is preceded by two non-coding exons. The protein-coding segment of the gene is located in exon 3, spanning residues 9422-10126. The amino acid sequence of FGF-BP1 is shown in SEQ ID NO: 2.

| SEQ ID NO: 1 (containing genomic sequence of FGF-BP1). | |
|---|---|
| Position of SNP indicated by “n” at position 184) | |
| ttgggaaaga tatggccttc caaatggaca tggcccttac tcagaagttt atcacaaaaa | 60 |
| taacaccacc ccagacaaca tcacctctta atttgatgtc aaaatgtcgg agtgatgagg | 120 |
| tatcaccact acagcaatca tcaaagtgtg agccatagac tcacctggtc aggtctagga | 180 |
| aaangagcca aaccatgttc atcttatctt cccttgagtg cctgatactt caccttgctg | 240 |
| agagtggtta ctgaataatg actgaagaga taacatcccc tttcccccac tgttgagctg | 300 |
| tgtttgcata attgcaggct cttgagaaat tcttgtctgc ctctggtagg ctagatgtcc | 360 |
| caactcaaag caccacagct ttccctccaa catagtcatt aacctctgtg tggtcatgac | 420 |
| tgatatcctt gacgtttccc ctattgtctg ctaactcctc aagggtaagg accatttttc | 480 |
| ttgactgcac ttccaggcaa tcattaacac tgagggcctg gacatagtag gtgcttcatg | 540 |
| cgtgctttta ggatgaatga aaaaatgact gagtggtagt agaaatgagg tcgcattagg | 600 |
| gtccagcctt gcagagtctc attcccctgc tcattcttgt cttctcctaa gcagcatgcc | 660 |
| acatgtgtga gtccttttgc tctttgtgta agggcattca gcacagaagg gtatctttct | 720 |
| gtgaggtgag gtcctccttg aacactccat cacgggttga cattcactgt tgggcccctg | 780 |
| agcattagga ccctctgaag atactcaccc atttgattcc aaatttggaa tcaaatttgg | 840 |
| tatccttcag cgctgaatgc ccttacaggt cttttcagag gtgcttttct ggtttgattt | 900 |
| ttctaaatat tgtcatctct tcatgtgaag aaagggatgt ggtcataagc caatgtgcct | 960 |
| cgtgactctc agccttaaca ccaagaaagg gcagcttacc ttctcccttt ttcagctttc | 1020 |
| aaagggagca actgttgctc tttgtagcac acatgggcta gaggctgaaa atccaaaagg | 1080 |
| gagactctct aggtccttct ttttctccaa gataggaagg gaggtagagg tggcatacag | 1140 |
| aaatgtgtac ttgtgtgggg ttttgtgatg tgtgagcatg tgtgtgggaa tgggtgtgta | 1200 |
| tatgtgagtg ctttcttaag tgcatgtatg tgggggtacc ttgaaaaatg ttgtccaaaa | 1260 |
| gacacagacc tctttaaagg gtgtttgact gtttaagaat gatttactta aaaacaaaga | 1320 |
| tccaaaaagc aaagaagtga tcaaaaaact tgatttgtag gggcaattga ttatagaaga | 1380 |
| gctaaaaaaa aaaaactttt acaaaaagaa aacactgtaa aaggctttga aatagcccta | 1440 |
| ttacttgaga aaaaatacct attattatta agtggtttca ttcaaatggc tgatattcta | 1500 |
| gcatttttga catactacaa atagtagttt cgtatgattt aacctaacac aaaaattgga | 1560 |
| gttaggtttc aaatgtgttg agaatcagaa ccaaccatta aacattgttt ctaaaaacat | 1620 |
| gttgatatga ttccactgct gggtatatac ccaaatgaaa atcattctac aaaaaagtaa | 1680 |
| ataaataaaa atttaaaaaa acaatatgca ctcatatgtt cattgtggta atattcacaa | 1740 |
| cagcaaaaac atggaatcaa cctggatgcc catcaaaggt gaattgacaa aagaaaatgt | 1800 |
| ggtacatata cattatggga tactatctat gcagccacga aaaataatga attgtgtcct | 1860 |
| ttgcagtaac atggatgcag ctggaggcta ttatcctaaa caaattaata caggaataga | 1920 |
| aaaccaaata ctgcatgttc tcacttataa gagaaggagc tagaccgagc acagtggctc | 1980 |
| acgactataa tcccaacact ttgggaggcc gaggcaggcg gatcatgagg tccggagttc | 2040 |
| gagagcagcc tggccaccat ggtgaaaccc cgtgtctact aaaaataaaa aaattagcca | 2100 |
| ggcatggtgg caggtgcctg taatcccagc tgctcggaag gctgaggcag gagaactgct | 2160 |
| tgaagccaga aggtggaggt tgcagtgagc caagatcgtg ccactgcact ctagcctggg | 2220 |
| tgacagagca agactttgtc tcggaaagaa aaaaaaaaga gaaggagcta aatattaggt | 2280 |
| aagcatggac ataaagatgg caaaaatagg gctggagact actagagtgg ggagaaatgg | 2340 |
| aggggggcaa ggttagaaaa actacctact gggtactatg ctcactacct gagtgataag | 2400 |
| atcatttgta cagcaaacct cagcgacatg caatttaccc atgtaacaaa tctacacatg | 2460 |
| taccctctga acctaaaata aaagttaaga aatgtgttaa tattatgtgg ttaataacaa | 2520 |
| cattctttct aaagtaaata aacttaatag gttttcacct tgcaatcata cccaatacta | 2580 |
| tagaatccac actctgctat ctcttcattt tctaaagaaa taaaactttc attgtcctga | 2640 |
| acaggaatcc tgaaatctca tgatgaatga gcactttaaa tttggaatgc aaatattctt | 2700 |
| tttttttttg tctcactttg tcgcccaggc tggagtgcag tggcacatct cggctcactg | 2760 |
| caagctccgc ctcctgggtt cacgccattc tcctgcctcg gcctcctgag cagctgggac | 2820 |
| tacaggcacc cgccaccatg cctagctaat ttttgtgtgt gtgtgtgtgt ttttagtaga | 2880 |
| gacagggttt caccatgtta gccaggatgg tctcgatctc ctgacctcat gatccgcctg | 2940 |
| ccctggcctc ccagaatgca aatattcttg agtaggtcta agattaatgc tttttgccag | 3000 |
| atgaattaga ttttactgat gtttgcttag cccaatttcc ttattttctg ataataaatg | 3060 |
| tattttaatg cacattattt gcccaacaaa ttaatattgc tttagtgcag cagctcagca | 3120 |
| tttgcaatgt ctgtttgtaa agtaacgttt gaaggaaggg ttgatttttg acaactttgg | 3180 |
| cctggcaata agtttggcac agttaagact tacctcagaa ccaaattgca cccattatat | 3240 |
| gaataatctc tggtgactgc ataagtaggg ccaggcctct aggtcaagaa gctatgtggt | 3300 |
| agtaacaggc atgagagtaa aatcagcttt tgaacttcaa atccccagct ggtgaccaga | 3360 |
| catgcattca attaccagcc ttagccagtg tgtttttacc aattgtgttc agacttcctt | 3420 |
| tcacaatccc aaagaaagta ttcatcattt aagttttgtg tgatcatgag aagaataaca | 3480 |
| tgatcccaat ctaaccagaa catgccagac aatttccaaa ttaaggacat tctcaaaaca | 3540 |
| cctaaccaat tcttctgcaa actgccatag tcatcaaaaa caagcaacat ctgagaaaca | 3600 |
| gccatagcca agaagaaccc aaaaagtcac gatcactaaa tgcaacctgg tgtcatgttg | 3660 |
| ggatcctgga cagaaaaaga acattaggag ctgaaaattt aattctacac caaacctgca | 3720 |
| catgcatgtt tatagcagct ctattcatca ctgtcaaaat ttagaggcat ccaaaatgtc | 3780 |
| ctagatgaat gaacaaatga actcgggtac atccaatcaa tggaatttta gtagtaggtg | 3840 |
| aatgaataaa gggacttgga tatatccaat ctttggaatt ttatcagcac ttaagagaaa | 3900 |
| tgtgctatca agttatgaaa aacatagaga aaccttaagt gcatattact aggtaaaaaa | 3960 |
| gtcaatataa aaggttacat attgtgtgat tccaactatt tgaaattctt aaaaagataa | 4020 |
| aactgaggag acagcaaaag gatgagcatt gcctggggtt gtaggggatg gagtggtaag | 4080 |
| taggcagaac acagagaatt tttagggcag ctaaactatt ctgtatgata ccatagtggt | 4140 |
| gggtaccgtc attatacctt tcttagttaa ttgatattat gcatgtcatt atacatttct | 4200 |
| caaaacccaa agaatgtaca gcacttagag tgaacctaat ggaaatgatg gacattgggt | 4260 |
| gataatgaca tgttaatgta ggtttattac ttacagcaaa tgtactaccc tggttcatga | 4320 |
| tgttgaaggt aggggagact gtgcctgcgt gtggggaggg gtatatggga cttttgatac | 4380 |
| tttccactca attgtgctat gagcataaaa ctactctaaa aaataaagtc tgtcaataat | 4440 |
| aataataata ataataaaaa ctagaacaac aggtaaatcc taagcaagtc tgaataaagt | 4500 |
| atggacttca attgacaata atgtatcaat gtttgtcagt tgtgacaaac gtaccatagt | 4560 |
| aatgtaacag gtaaataggg aacccaggtg agaattggaa tgataactat ctttgcaatt | 4620 |
| ctctgtaaac ctaaaattat tctaaaactt taaaaaaaaa tttaaaggga aggtatgaag | 4680 |
| gggcaaagag aggagaattc cacaatttat cagtgtatgc gtacactgcc tcatttgtgt | 4740 |
| ttagaatgat agcagcggcc tccagataat tcttctctaa ttgtgtgtca gacactgtgc | 4800 |
| ttagtccccc taagccttgc ctccacatct gtcccatgag gacctgggcc gactttcctc | 4860 |
| ctggggttct tgtgcaatgg gcctttatgt actcatcccc aagaattaca gagcccctca | 4920 |
| gtgctgggag catggggagg tggaggggcc tgaagttact ggaacctgct atgcactgcg | 4980 |
| tggctggggc tccatgtcct ctgcagtctc cccagagcca ctggaaaagg acatcgtttc | 5040 |
| cttagttcat ggagatacga agaaagcttg ggaaacagcc ttagcacagt gcctggcacg | 5100 |
| ctgtaagctc attggaggaa gtgtgccatt tctccccgtg gacattctta aagatgtggg | 5160 |
| tggcatgcag ggcgtcagga gccaatgaca ccaacagaga ggggctcgca ttggagcctg | 5220 |
| gccttgaagg atgaggcggt gtgtgccagg cagagaagga agggaagggg atcccaagtg | 5280 |
| cagggaacag cagaggcaaa ggcaggaagt gggcgcatgc atggagtatc tgcaaatggt | 5340 |
| gggacagggt ttcagtgtgg ccagattgca gtgtgtgtgt gcatgtgtgt gtgtgtctgt | 5400 |
| ctctgtgtgt gtgtcagtgt cactgtgtgt gattctgtgt gtgtgtgcct atgagtatgt | 5460 |
| gtgtgaatgt gtctaggtgt cagtatgtgt gtgtctctgt gtgtctatga gtgtggtgtc | 5520 |
| tttgagtgtg tttgtgtgtg tttgagtgtg tcagtgtgtg tttgtgtgtg tctttgtatg | 5580 |
| tgtgtcagtg tgtatgtgta tttatgtgtc tgcgtgtgag tacctggaaa tgaggttgga | 5640 |
| agggcaggat gagccaagga ggtgactgcc ccacagcctg ctctgatgct gtgctgctga | 5700 |
| gtctagaagg gaaagagcaa agcagacgct gggatgggag tcacaggaag gggaagggcc | 5760 |
| acaaggagcc agtggggaat gtgcagtgtt acctggcaaa gctcagagaa gcacttccca | 5820 |
| gaaagacccc ggagtccaca tgtttccttg taaatatgtt ttgtgttcca actgcaatct | 5880 |
| gcctccagaa agaatctgcc tgcctcccaa ggatcaagta gaattttcca ttttgtagga | 5940 |
| gactcccctt gttgagcaaa gcagctttgc cttgcctatg tccaaataat cctccaaaat | 6000 |
| accgcatgag agagcctgtt cgtggaggtc agggcctcag cggggtgttt gtgaggatag | 6060 |
| tgaggacgga cccctgggtg aggcaatttt aggagagagg cacacagctt cagaaatggc | 6120 |
| ttcttccaac agattcatgc agagactgag cctctgagta aattggtcaa catctatagc | 6180 |
| tatgaccacg gaattctcaa acaggtggat cgttcataaa atccaaacac agttacaagt | 6240 |
| tttggcctgc atagtgttga gctacagctt tatttttaga aaattttaat tagctgccaa | 6300 |
| ccaatttcat ataaacacat ctggatttgg ggatgtcatt ttaaaaatca gacagtgtga | 6360 |
| caatactgag cctaatccct gcattttctc agtcaattgg agatgagtgt tgacctctat | 6420 |
| ggaggcagag actctctagt ttgccacagt ccccatcact ccctattgcc ttatctcaaa | 6480 |
| cccaaaagtc agttgccatt taatatcaca ctaaacaata gttttcttat ggctgaatta | 6540 |
| aggaaaagtg aacaatttct tcttctgaaa atctatgaaa ggtgaggtga taatatataa | 6600 |
| tgagggccac atcttccctg tagaagtgag tttcttttta aatgctgcat atgtaaataa | 6660 |
| caaatgtctg ttttcaaagt atacaactta agaccctgtc atgaggcaaa acgattctgc | 6720 |
| tccactttat gttaacagct tagcccagta aaaatttagt atgcagctat aaaggagacg | 6780 |
| ttgttattgt cagcacactg caaatggatg atgggagagg ctaagtggtt tgggcatagc | 6840 |
| actcagctaa acacatgctg tggccaggat tccacctggg tcattctggt gtgggactgt | 6900 |
| gcctgtagca ggcagggcag cctgagctct ctgaatgaga cctgaaggta agtatttaca | 6960 |
| acagtaatct tatattaaaa attccctctt taccagcctc tcagagcctg gtagaattcc | 7020 |
| ctgataaaaa agagggaatt tttaatgtaa atttccctgc ctctagaatt agggtatcct | 7080 |
| ctgcttatgt agtaacttca gatggaaaaa ggaactcaga cactacgaaa acttcggcaa | 7140 |
| cttcagtcac ccattcaaca aagatttatt gagagtggca ggcaggaaga gatcagacca | 7200 |
| tggatgtaga gaatctcact tttaattttg agatgttcct tttcctgcct ctgtgaactt | 7260 |
| ggacaagctc ttacatatct ccagaaccaa ttcctgcttt tgtaaaaggg gaggaatcat | 7320 |
| tgcactccct actagagcgg atgtgatgag ggaaaaggag aactcaggta aaatgggctc | 7380 |
| agtagtttgg cagctcggat catgtcatta tagttatttt actcctccat agacatcact | 7440 |
| gctggggtcc tctccccatc aaggagggtc actgtgaggc tgagttgtcc tcctatcctg | 7500 |
| gccacagggt ggacagggaa gggcctagag gaagcgtttg atggcatgca ggtaaccccc | 7560 |
| cttttcactg gattacatcc agcactttcc ctgcaggaac cggctccctc ggaggggcgt | 7620 |
| ggctgggagg agctgtgagt aacgtgccac agtgttgtaa aaacccagtg agtgttataa | 7680 |
| aaacccagtc agcctggctc ctgttgaata gtctaccccc cttgcactct acctgacaca | 7740 |
| gctgcagcct gcaattcact cgcactgcct gggattgcac tggatccgtg tgctcagaac | 7800 |
| aaggtaagag cctcgctcct ttaacagctt tattacctgc tgccacttct cactccacca | 7860 |
| agggcctgga gttccttggg cctgcattcc atcatgggca ggtgggggct gagccccagg | 7920 |
| gccgtgagaa atgactgggt gtttttctgg catatccagt ctcctcttca ccatttattg | 7980 |
| ctgttctctg cccttttgtg attgtctttc tccttcttaa ccctagaaga tggccaactt | 8040 |
| ctagggtaga aatcagatca ggaccatttc ctgggtctgc ctctttttat aaaaaattct | 8100 |
| gtgttttgtt ttaaatggag cacaaataat acaaatcatc ttatcattat gtgttatatg | 8160 |
| aaactaaatt gtcattgttc atagttgttt ttcttattct ttggcaatgt ttttaaaggg | 8220 |
| cagagtaaat gtgaaaagca aaaagtgatc aaccaggggt gacagaaagg aataaggaga | 8280 |
| ttgatctggc tacccatacc agctttgaac ttgcccacta tgagatcctg agcaatttac | 8340 |
| ttacctgtct gagccttaag agtcagagct gtcacctggg atgacatatt aatagctacc | 8400 |
| tggaagagct cttgtgtgga tttcacaagg tgggaatcaa accctagacc agtacttagc | 8460 |
| acacaggagg tgtcatatag aaactgactt ctcttcttct gttaagcatg ggtttttatt | 8520 |
| gtacacatga tcagattcat gcagttcagt gtgacgttgg tgttcatcct attgattcaa | 8580 |
| tgatttctca tgatccttgt cttcgatctt gtagtgtgaa ctatctggtt gaaattcacc | 8640 |
| tctctccacc ctgattcact ttgcccaggc aaatcagaca accaccccac ctcacacctt | 8700 |
| tccttaaact cttacttccc cacaaaaagc tcccccaccc ccacaacccc acccacccca | 8760 |
| cccaaacccc aggcatgttc aactctgggt caacatcacc tttcatgatg cttaccacat | 8820 |
| cctttctcat tttgggcatc tgtcatgtct tttctcattt tgggcatctg tgtccatatg | 8880 |
| gagtcatgag atagaaatcc agccctttgg agctgaaagt ttggagcagt aacttgtttc | 8940 |
| atatcagaaa cagagatgac gggatagatt ccctgatcaa tgctatttaa attctcccaa | 9000 |
| agagtggtct ctaagctctt tcaaatctga ggctttctca atcatggtga tttcttcaac | 9060 |
| acctagtctg gtggggggca tatagtaggt gctcagtaaa tgcttgttga atgagtgaat | 9120 |
| gaaagagtga ggagtgagca gatgcaggag ttaatgagtg aataaatgat aaatgagagt | 9180 |
| tcgtgaggct ccaaagagtg gtacttttgt gacactcaag actcaagtca agtggaaatg | 9240 |
| tgagtagaca ggctgtttgt ctagtgactc caggagccag aagcgttctt ggtctactct | 9300 |
| gaaatcatgt aggcacctgt cagggtaggg ggaggccagc gtgtagtcct gcacagctga | 9360 |
| actgctgcct gccactgact cacttgcctt ttgtcaaaca ggtgaacgcc cagctgcagc | 9420 |
| catgaagatc tgtagcctca ccctgctctc cttcctccta ctggctgctc aggtgctcct | 9480 |
| ggtggagggg aaaaaaaaag tgaagaatgg acttcacagc aaagtggtct cagaacaaaa | 9540 |
| ggacactctg ggcaacaccc agattaagca gaaaagcagg cccgggaaca aaggcaagtt | 9600 |
| tgtcaccaaa gaccaagcca actgcagatg ggctgctact gagcaggagg agggcatctc | 9660 |
| tctcaaggtt gagtgcactc aattggacca tgaattttcc tgtgtctttg ctggcaatcc | 9720 |
| aacctcatgc ctaaagctca aggatgagag agtctattgg aaacaagttg cccggaatct | 9780 |
| gcgctcacag aaagacatct gtagatattc caagacagct gtgaaaacca gagtgtgcag | 9840 |
| aaaggatttt ccagaatcca gtcttaagct agtcagctcc actctatttg ggaacacaaa | 9900 |
| gcccaggaag gagaaaacag agatgtcccc cagggagcac atcaaaggca aagagaccac | 9960 |
| cccctctagc ctagcagtga cccagaccat ggccaccaaa gctcccgagt gtgtggagga | 10020 |
| cccagatatg gcaaaccaga ggaagactgc cctggagttc tgtggagaga cttggagctc | 10080 |
| tctctgcaca ttcttcctca gcatagtgca ggacacgtca tgctaatgag gtcaaaagag | 10140 |
| aacgggttcc cttaagagat gtcatgtcgt aagtccctct gtatacttta aagctctcta | 10200 |
| cagtcccccc aaaatatgaa cttttgtgct tagtgagtgc aacgaaatat ttaaacaagt | 10260 |
| tttgtatttt ttgcttttgt gttttggaat ttgccttatt tttcttggat gcgatgttca | 10320 |
| gaggctgttt cctgcagcat gtatttccat ggcccacaca gctatgtgtt tgagcagcga | 10380 |
| agagtctttg agctgaatga gccagagtga taatttcagt gcaacgaact ttctgctgaa | 10440 |
| ttaatggtaa taaaactctg ggtgtttttc agaaatacat tcaaacattg cttggttttc | 10500 |
| tctgtgtctg gtcccaaata aactcattaa gtagtcccca tatatcaatg gaaaaacaat | 10560 |
| ctaaacagga acttccagtt ccccaccctt cgcaagttat ctctt | 10605 |
| SEQ ID NO 2 (amino acid sequence of FGF-BP1) | |
| Met Lys Ile Cys Ser Leu Thr Leu Leu Ser Phe Leu Leu Leu Ala Ala | |
| 1 5 10 15 | |
| Gln Val Leu Leu Val Glu Gly Lys Lys Lys Val Lys Asn Gly Leu His | |
| 20 25 30 | |
| Ser Lys Val Val Ser Glu Gln Lys Asp Thr Leu Gly Asn Thr Gln Ile | |
| 35 40 45 | |
| Lys Gln Lys Ser Arg Pro Gly Asn Lys Gly Lys Phe Val Thr Lys Asp | |
| 50 55 60 | |
| Gln Ala Asn Cys Arg Trp Ala Ala Thr Glu Gln Glu Glu Gly Ile Ser | |
| 65 70 75 80 | |
| Leu Lys Val Glu Cys Thr Gln Leu Asp His Glu Phe Ser Cys Val Phe | |
| 85 90 95 | |
| Ala Gly Asn Pro Thr Ser Cys Leu Lys Leu Lys Asp Glu Arg Val Tyr | |
| 100 105 110 | |
| Trp Lys Gln Val Ala Arg Asn Leu Arg Ser Gln Lys Asp Ile Cys Arg | |
| 115 120 125 | |
| Tyr Ser Lys Thr Ala Val Lys Thr Arg Val Cys Arg Lys Asp Phe Pro | |
| 130 135 140 | |
| Glu Ser Ser Leu Lys Leu Val Ser Ser Thr Leu Phe Gly Asn Thr Lys | |
| 145 150 155 160 | |
| Pro Arg Lys Glu Lys Thr Glu Met Ser Pro Arg Glu His Ile Lys Gly | |
| 165 170 175 | |
| Lys Glu Thr Thr Pro Ser Ser Leu Ala Val Thr Gln Thr Met Ala Thr | |
| 180 185 190 | |
| Lys Ala Pro Glu Cys Val Glu Asp Pro Asp Met Ala Asn Gln Arg Lys | |
| 195 200 205 | |
| Thr Ala Leu Glu Phe Cys Gly Glu Thr Trp Ser Ser Leu Cys Thr Phe | |
| 210 215 220 | |
| Phe Leu Ser Ile Val Gln Asp Thr Sen Cys | |
| 225 230 | |

### SUMMARY OF THE INVENTION

The present invention identifies an association between COPD and the FGF-BP1 gene. More particularly, the present application identifies a single nucleotide polymorphism (SNP) within a putative regulatory region of this gene which is associated with COPD. The present application therefore provides direct evidence of a role for FGF-BP1 in COPD. Thus, the FGF-BP1 gene (including its regulatory regions), mRNA and the FGF-BP1 polypeptide may act as a diagnostic or prognostic marker of COPD, and can be used to design specific probes, or to generate antibodies, capable of detecting the presence of single nucleotide polymorphisms (SNPs) or mutations of the gene or mRNA, or for measuring the levels of the mRNA or encoded protein present in a test sample, such as a body fluid or cell sample. In addition the FGF-BP1 gene and the protein encoded thereby is a potential target for therapeutic intervention in COPD, for instance in the development of antisense nucleic acid targeted to the mRNA; or more widely in the identification or development of chemical or hormonal therapeutic agents. The person skilled in the art is also capable of devising screening assays to identify compounds (chemical or biological) that modulate FGF-BP1, which compounds may prove useful as therapeutic agents in treating or preventing COPD.

### DETAILED DESCRIPTION OF THE INVENTION

The following definitions apply to the present application.

“Allele” refers to a particular form of a genetic locus, distinguished from other forms by its particular nucleotide or amino acid sequence.

“Expression” refers to the transcription of a gene's DNA template to produce the corresponding mRNA and translation of this mRNA to produce the corresponding gene product (i.e., a peptide, polypeptide, or protein). The term “activates gene expression” refers to inducing or increasing the transcription of a gene in response to a treatment where such induction or increase is compared to the amount of gene expression in the absence of said treatment. Similarly, the terms “decreases gene expression” or “down-regulates gene expression” refers to inhibiting or blocking the transcription of a gene in response to a treatment and where such decrease or down-regulation is compared to the amount of gene expression in the absence of said treatment.

“Functional activity” of a protein in the context of the present invention describes the function the protein performs in its native environment. Altering the functional activity of a protein includes within its scope increasing, decreasing or otherwise altering the native activity of the protein itself. In addition, it also includes within its scope increasing or decreasing the level of expression and/or altering the intracellular distribution of the nucleic acid encoding the protein, and/or altering the intracellular distribution of the protein itself.

“Gene” is a segment of DNA that contains all the information for the regulated biosynthesis of an RNA product, including a promoter, exons, introns, and other sequence elements which may be located within 5′ or 3′ flanking regions (not within the transcribed portions of the gene) that control expression.

“Genotype” is an unphased 5′ to 3′ sequence of nucleotide pair(s) found at one or more polymorphic sites in a locus on a pair of homologous chromosomes in an individual.

“Isolated” nucleic acid, as referred to herein, refers to material removed from its original environment (for example, the natural environment in which it occurs in nature), and thus is altered by the hand of man from its natural state. For example, an isolated polynucleotide could be part of a vector or a composition of matter, or could be contained within a cell, and still be “isolated” because that vector, composition of matter, or particular cell is not the original environment of the polynucleotide.

“Locus” refers to a location on a chromosome or DNA molecule corresponding to a gene or a physical or phenotypic feature.

“Nucleic acid”, as used herein, refers to single stranded or double stranded DNA and RNA molecules including natural nucleic acids found in nature and/or modified, artificial nucleic acids having modified backbones or bases, as are known in the art.

“Polymorphic site” is a position within a locus at which at least two alternative sequences are found in a population.

“Polymorphism” refers to the sequence variation observed in an individual at a polymorphic site. Polymorphisms include nucleotide substitutions, insertions, deletions and microsatellites and may, but need not, result in detectable differences in gene expression or protein function.

“Stringent hybridisation conditions” refers to an overnight incubation at 42° C. in a solution comprising 50% formamide, 5×SSC (750 mM NaCl, 75 mM trisodium citrate), 50 mM sodium phosphate (pH 7.6), 5×Denhardt's solution, 10% dextran sulphate, and 20 pg/ml denatured, sheared salmon sperm DNA, followed by washing the filters in 0.1×SSC at about 65° C.

“Variant” or “derivative” in relation to the FGF-BP1 gene or polypeptide includes any substitution of, variation of, modification of, replacement of, deletion of or addition of one (or more) nucleic or amino acids from or to the nucleotide or polypeptide sequence of FGF-BP1.

The present invention identifies an association between COPD and the FGF-BP1 gene. More particularly, the present application identifies a single nucleotide polymorphism (SNP) within a putative regulatory region of this gene which is associated with COPD. The present application therefore provides direct evidence for a role for FGF-BP1 in COPD, and is in accordance with further in house studies which have shown that expression of FGF-BP1 mRNA is increased in patients with COPD and further increases with severity of COPD.

Polymorphisms can help identify patients most suited to therapy with particular pharmaceutical agents (this is often termed “pharmacogenetics”). Pharmacogenetics can also be used in pharmaceutical research to assist the drug selection process. Polymorphisms are used in mapping the human genome and may be used to elucidate the genetic component of diseases. The reader is directed to the following references for background details on pharmacogenetics and other uses of polymorphism detection: Linder et al. (1997), Clinical Chemistry, 43, 254; Marshall (1997), Nature Biotechnology, 15, 1249; International Patent Application WO 97/40462, Spectra Biomedical; and Schafer et al. (1998), Nature Biotechnology, 16, 33.

Single nucleotide polymorphisms (SNPs) are single base pair positions in genomic DNA at which different sequence alternatives (alleles) exist in normal individuals in a population. It is recognised that SNPs may be responsible for variations between individuals, including variations which predispose an individual to a disease or cause it. Approximately half of all coding sequence SNPs result in synonymous (i.e. silent) codon changes. Even though these SNPs may have no effect on protein function, they are potentially useful for tracking other variations nearby as adjacent stretches of DNA tend to be inherited together (‘linkage disequilibrium’).

Clinical trials have shown that patient response to treatment with pharmaceuticals is often heterogeneous. Thus, there is a need for improved approaches to pharmaceutical agent design and therapy.

In the light of the above, the present invention identifies an association between COPD and a polymorphism in the 5′ flanking region of the human FGF-BP1 encoding gene. The associated polymorphism is a SNP located in SEQ ID NO: 1 which is 7131 bp upstream of exon 1. At the position of the SNP, the nucleotide base most often found is a G (for guanine) but in about 15% of chromosomes (of Caucasian ethnicity) the base is an A (for adenine). Chromosomes that have an “A” nucleotide at the position of the SNP contain a putative binding site for nuclear factors of activated T cells (NFAT) (consensus sequence [T/A]GGAAAA[A/T]) while this site is absent on chromosomes bearing the “G” allele. Without being bound by theory, the consequence of a SNP being located at the position of a putative transcriptional regulatory element is that the level of expression of the neighbouring gene may depend upon which allele is present. Our genetic study (see below) indicated that the “A” allele is more likely to be found among COPD cases than controls. One possible explanation for this observation is that on chromosomes bearing the “A” allele, the FGF-BP1 gene is up-regulated (as a consequence of the NFAT site), increasing the risk for COPD.

Thus, according to one aspect of the present invention there is provided a method for assessing the predisposition and/or susceptibility of an individual to COPD, which method comprises

(a) determining the identity of one or more nucleotides at a polymorphic site in an FGF-BP-1 gene of the individual; and
(b) determining the status of the individual by reference to said one or more nucleotides.

The polymorphic site will be one which has an association with COPD in a human population. By this is meant that a particular nucleotide or nucleotide sequence at the polymorphic site is correlated with incidence of COPD or with a related phenotype, such as bronchitis, chronic airway obstruction or severity of disease, and occurs at a greater frequency in susceptible patients (for example a site which contains e.g. a single nucleotide substitution, nucleotide insertion and nucleotide deletion which occurs with greater frequency in COPD patients/COPD susceptible subjects). Methods of identifying such sites in the FGF-BP1 gene are described herein with reference to SNPs. However, the same principles can be used for other polymorphisms. Furthermore, methods to identify nucleotides at such sites in the FGF-BP1 gene are described herein. In this way, the individual can be genotyped with respect to the particular polymorphic site. The polymorphic site may correspond to a polymorphism selected from a single nucleotide substitution, nucleotide insertion and nucleotide deletion which in the case of insertion and deletion includes insertion or deletion of one or more nucleotides at a position of a gene. Preferably, the polymorphism is a single nucleotide polymorphism. The method of the present invention may involve determining the identity of one or more nucleotides at two or more polymorphic sites in the FGF-BP1 gene.

A particularly strong association has been found between COPD and a single nucleotide polymorphism in the 5′ flanking region of the human FGF-BP1 gene. This single nucleotide polymorphism is defined as that corresponding to position 184 of SEQ ID NO:1.

Accordingly, the present invention also provides a method for assessing the predisposition and/or susceptibility of an individual to COPD, which method comprises determining the nucleotide of the individual at position 184 of SEQ ID NO:1.

In a further embodiment, the method comprises detecting for the presence or absence of A and/or G at position 184 of SEQ ID NO:1.

It should be noted that in this application, SNPs are referred to by reference to a position in SEQ ID NO:1 (e.g. position 184). However, when such references are made, it will be understood that the invention is not to be limited to the exact sequence as set out in that listing but includes variants and derivatives thereof. Thus, identification of SNP locations in similar sequences are contemplated (i.e. SNPs at positions which the skilled person would consider correspond to the positions identified in the SEQ ID numbers). The person skilled in the art can readily align similar sequences and locate the same SNP locations. The position of the SNPs refers to the position in SEQ ID NO: 1 where the first nucleotide in the sequence listed is position 1.

It should further be noted that detection of the nucleotide in the complement strand to SEQ ID NO:1 that base-pairs with the nucleotide at position 184 of SEQ ID NO:1 is of course within the scope of the claimed invention.

The term individual in the context of the present invention means human, and includes a human having or suspected of having COPD and an asymptomatic human who may be tested for predisposition or susceptibility to such a disease.

The method for determining the nucleotide(s) at a polymorphic site in an FGF-BP 1 gene may be, for example, a method selected from amplification refractory mutation system, sequencing, allelic discrimination assay, hybridisation, restriction fragment length polymorphism, oligonucleotide ligation assay, or allele specific PCR.

Methods for determining the sequences of nucleic acid sequences and determining the identity of nucleotides at particular positions within a sequence will be recognised to those skilled in the art and suitable methods are described herein. A number of these methods employ binding an oligonucleotide probe to a nucleic acid sample derived from an individual. The probe may comprise a nucleotide sequence which binds specifically to a particular allele of one of the polymorphisms whilst not binding specifically to other alleles of the polymorphisms.

The test sample of nucleic acid is conveniently a sample of blood, mouth swab, biopsy, or other body fluid or tissue obtained from an individual. It will be appreciated that the test sample may equally be a nucleic acid sequence corresponding to the sequence in the test sample, that is to say that all or a part of the region in the sample nucleic acid may firstly be amplified using any convenient technique e.g. PCR, before analysis of allelic variation. In one embodiment of the invention, the detection of the polymorphism is determined from a nucleic acid sample (which may be as defined above) that has already been removed from the individual. Therefore, in each aspect of the invention where the analysis of nucleic acid is required, the invention includes the case where the nucleic acid sample has already been removed from the individual.

Thus, the present invention also provides a method for assessing the predisposition and/or susceptibility of an individual to COPD, which method comprises:

(a) providing a nucleic acid sample that has been removed from the individual;
(b) determining the identity of one or more nucleotides at a polymorphic site in an FGF-BP-1 gene of the individual; and
(c) determining the status of the individual by reference to said one or more nucleotides.

Again, the polymorphic site will be one which has an association with COPD in a human population. In this way, the individual can be genotyped with respect to the polymorphic site. An individual's genotype is the unphased 5′ to 3′ sequence of nucleotide pairs found at one or more polymorphic sites in a locus on a pair of homologous chromosomes in an individual. Accordingly, the present invention also includes a method for assessing the predisposition and/or susceptibility of an individual to COPD by genotyping an individual comprising determining the identity of the nucleotide pair (or nucleotide sequence pair) at the polymorphic sites identified herein in the two copies of the FGF-BP-1 gene.

It will be apparent to the person skilled in the art that there are a large number of analytical procedures which may be used to detect the presence or absence of variant nucleotides at one or more polymorphic positions of the invention. In general, the detection of allelic variation requires a mutation discrimination technique, optionally an amplification reaction and optionally a signal generation system. Table 1 lists a number of mutation detection techniques, some based on the PCR. These may be used in combination with a number of signal generation systems, a selection of which is listed in Table 2. Further amplification techniques are listed in Table 3. Many current methods for the detection of allelic variation are reviewed by Nollau et al., Clin. Chem. 43, 1114-1120, 1997; and in standard textbooks, for example “Laboratory Protocols for Mutation Detection”, Ed. by U. Landegren, Oxford University Press, 1996 and “PCR”, 2^{nd} Edition by Newton & Graham, BIOS Scientific Publishers Limited, 1997.

Abbreviations:

| | |
|---|---|
| ALEX ™ | Amplification refractory mutation system linear extension |
| APEX | Arrayed primer extension |
| ARMS ™ | Amplification refractory mutation system |
| b-DNA | Branched DNA |
| bp | base pair |
| CMC | Chemical mismatch cleavage |
| COPS | Competitive oligonucleotide priming system |
| DGGE | Denaturing gradient gel electrophoresis |
| FRET | Fluorescence resonance energy transfer |
| HMG-CoA | 3-hydroxy-3-methylglutaryl-coenzyme A |
| LCR | Ligase chain reaction |
| MASDA | Multiple allele specific diagnostic assay |
| NASBA | Nucleic acid sequence based amplification |
| OATP | Na+-independent organic anion transporting polypeptide |
| OLA | Oligonucleotide ligation assay |
| PCR | Polymerase chain reaction |
| PTT | Protein truncation test |
| RFLP | Restriction fragment length polymorphism |
| SDA | Strand displacement amplification |
| SNP | Single nucleotide polymorphism |
| SSCP | Single-strand conformation polymorphism analysis |
| SSR | Self sustained replication |
| ALEX ™ | Amplification refractory mutation system linear extension |
| TGGE | Temperature gradient gel electrophoresis |

### Examples of Mutation Detection Techniques Include:

General: DNA sequencing, Sequencing by hybridisation, Pyrosequencing™
Scanning: PTT*, SSCP, DGGE, TGGE, Cleavase, Heteroduplex analysis, CMC, Enzymatic mismatch cleavage
*Note: not useful for detection of promoter polymorphisms.

### Hybridisation Based

Solid phase hybridisation: Dot blots, MASDA, Reverse dot blots, Oligonucleotide arrays (DNA Chips).
Solution phase hybridisation: Taqman™—U.S. Pat. No. 5,210,015 & U.S. Pat. No. 5,487,972 (Hoffmann-La Roche), Molecular Beacons—Tyagi et al (1996), Nature Biotechnology, 14, 303; WO 95/13399 (Public Health Inst., New York).
Extension Based: ARMS™, ALEX™—European Patent No. EP 332435 B1 (Zeneca is Limited), COPS—Gibbs et al (1989), Nucleic Acids Research, 17, 2347.

### Incorporation Based: Mini-sequencing, APEX.

Restriction Enzyme Based: RFLP, Restriction site generating PCR.

### Ligation Based: OLA.

Other: Invader assay.
Examples of Signal Generation or Detection Systems include:
Fluorescence: FRET, Fluorescence quenching, Fluorescence polarisation—United Kingdom Patent No. 2228998 (Zeneca Limited)
Other: Chemiluminescence, Electrochemiluminescence, Raman, Radioactivity, Colorimetric, Hybridisation protection assay, Mass spectrometry

Further Amplification Methods include SSR, NASBA, LCR, SDA, b-DNA.

Preferred mutation detection techniques include ARMS™, ALEX™, COPS, Taqman, Molecular Beacons, RFLP, and restriction site based PCR and FRET techniques.

It will be appreciated that the test sample may equally be a nucleic acid sequence corresponding to the sequence in the test sample, that is to say that all or a part of the region in the sample nucleic acid may firstly be amplified using any convenient technique e.g. polymerase chain reaction (PCR), before analysis. The nucleic acid may be genomic DNA or fractionated or whole cell RNA. In one embodiment the RNA is whole cell RNA and is used directly as the template for labelling a first strand cDNA using random primers or poly A primers. The nucleic acid or protein in the test sample may be extracted from the sample according to standard methodologies (Sambrook et al. “Molecular Cloning—A Laboratory manual”, second edition. Cold Spring Harbor, N.Y. (1989)).

It will be apparent that the gene sequence disclosed for FGF-BP1 (as depicted in SEQ ID NO: 1) is a representative sequence. In normal individuals there are two copies of each gene, a maternal and paternal copy, which will likely have some sequence differences, moreover within a population there will exist numerous allelic variants of the gene sequence. It will be appreciated that the diagnostic methods and other aspects of this invention extend to the detection etc. of any of these sequence variants. Preferred sequence variants are those that possess at least 80%, preferably 90% and more preferably at least 95% sequence identity (nucleic acid or amino acid) to FGF-BP1 depicted in SEQ ID No. 1 or 2. Nucleic acid sequence identity can also be gauged by hybridisation studies whereby, under stringent hybridisation and wash conditions, only closely related sequences (for example, those with >90% identity) are capable of forming a hybridisation complex.

In a further aspect, the methods of the invention are used to assess the pharmacogenetics of a drug capable of interacting with COPD.

Assays, for example reporter-based assays, may be devised to detect whether one or more of the polymorphisms according to the present invention affect transcription levels and/or message stability.

Individuals who carry particular allelic variants of the FGF-BP1-encoding genes may therefore exhibit differences in their ability to regulate protein biosynthesis under different physiological conditions and will display altered abilities to react to COPD. In addition, differences arising as a result of allelic variation may have a direct effect on the response of an individual to drug therapy. The diagnostic methods of the invention may be useful both to predict the clinical response to such agents and to determine therapeutic dose.

In a further aspect, the methods of the invention are used in the development of new drug therapies which selectively target FGF-BP1 or one or more allelic variants of FGF-BP1. Identification of a link between a particular allelic variant and predisposition to disease development or response to drug therapy may have a significant impact on the design of new drugs. Drugs may be designed to regulate the biological activity of variants implicated in the disease process whilst minimising effects on other variants.

In a further diagnostic aspect of the invention, the presence or absence of variant nucleotides is detected by reference to the loss or gain of, optionally engineered, sites recognised by restriction enzymes.

According to another aspect of the present invention there is provided an allele-specific oligonucleotide primer or an allele-specific oligonucleotide probe capable of detecting a polymorphism in the FGF-BP1 encoding gene (or its complimentary strand), and which polymorphism preferably corresponds to a position defined herein (or to a sequence complementary to such a polymorphic sequence).

Thus, the present invention provides an allele-specific oligonucleotide primer or an allele-specific oligonucleotide probe which is capable of detecting a polymorphism at position 184 of SEQ ID NO:1.

It should be noted that reference to an allele-specific oligonucleotide primer or an allele-specific oligonucleotide probe which is capable of detecting a polymorphism at position 184 of SEQ ID NO:1 includes an allele-specific oligonucleotide primer or an allele-specific oligonucleotide probe which is capable of detecting the compliment of a polymorphism at position 184 of SEQ ID NO: 1.

According to a further feature of this aspect, the present invention provides a primer or probe which is capable of detecting a polymorphism defined by the presence of G at position 184 of SEQ ID NO: 1. In a further embodiment, the present invention provides a primer or probe which is capable of detecting a polymorphism defined by the presence of A at position 184 of SEQ ID NO:1.

Each primer or probe of the present invention may be of any suitable length and will typically be 17-50 nucleotides in length, preferably about 17-35 nucleotides, more preferably about 17-30 nucleotides in length.

The allele-specific primers of the present invention are used, generally together with a constant primer, in an amplification reaction such as a PCR reaction, which provides the discrimination between alleles through selective amplification of one allele at a particular sequence position e.g. as used for ARMS™ assays.

For example, in one embodiment, an allele-specific primer capable of detecting a polymorphism at position 184 of SEQ ID NO: 1 should be able to discriminate, in an amplification reaction such as a PCR reaction, between an FGF-BP1 gene or a fragment thereof comprising base G at position 184 of SEQ ID NO: 1 (or a sequence complementary to such a gene or fragment), and an FGF-BP1 gene or a fragment thereof comprising base A at position 184 of SEQ ID NO:1 (or a sequence or fragment complementary to such a gene or fragment).

An allele-specific primer of the present invention preferably corresponds exactly with (or is completely complimentary with) the allele to be detected but derivatives thereof are also contemplated wherein e.g. 6 or more (e.g. 6 to 8) of the nucleotides at the 3′ terminus correspond exactly with (or are completely complimentary with) the allele to be detected and wherein up to 10, such as up to 8, 6, 4, 2, or 1 of the remaining nucleotides may be varied without significantly affecting the properties of the primer.

Primers may be manufactured using any convenient method of synthesis. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7; 1993; 1^{st} Edition. If required the primer(s) may be labelled to facilitate detection.

The primers and/or probes of the present invention will typically be in the form of nucleic acids (e.g. DNA or cDNA). Alternatively, the primers and/or probes may be in the form of nucleic acid analogues, for example PNA (Peptide Nucleic Acids) or LNA (Locked Nucleic Acids). The primers or probes may be nucleic acids which have been substituted in part by nucleotide analogues (such as LNA or PNA). By employing nucleic acid analogues, specific hybridisation can be achieved with shorter oligonucleotides down to 6 bases in length.

The design of such probes will be apparent to the molecular biologist of ordinary skill. Such probes are of any convenient length such as up to 50 bases, up to 40 bases, more conveniently up to 30 bases in length, such as for example 8-25 or 8-15 bases in length. In general such probes will comprise base sequences entirely complementary to the corresponding wild type or variant locus in the gene. However, if required one or more mismatches may be introduced, provided that the discriminatory power of the oligonucleotide probe is not unduly affected. The probes of the invention may carry one or more labels to facilitate detection.

In one embodiment, an allele-specific probe capable of detecting a polymorphism at position 184 of SEQ ID NO:1 can discriminate, in a hybridisation reaction, between a FGF-BP1 gene or a fragment thereof comprising base G at position 184 of SEQ ID NO:1 (or a sequence complementary to such a gene or fragment), and a FGF-BP1 gene or a fragment thereof comprising base A at position 184 of SEQ ID NO:1 (or a sequence or fragment complementary to such a gene or fragment).

According to another aspect of the present invention there is provided a diagnostic kit comprising an allele-specific oligonucleotide probe of the invention and/or an allele-specific primer of the invention.

Thus the kit may comprise an allele-specific oligonucleotide primer capable of detecting a polymorphism at position 184 of SEQ ID NO: 1.

Alternatively, the kit may comprise an allele-specific oligonucleotide probe capable of detecting a polymorphism at position 184 of SEQ ID NO: 1.

The diagnostic kits may comprise appropriate packaging and instructions for use in the methods of the invention. Such kits may further comprise appropriate buffer(s) and polymerase(s) such as thermostable polymerases, for example taq polymerase.

According to another aspect of the present invention there is provided a method of treating a human having, or at risk of having, COPD, with a drug capable of modulating FGF-BP1, which method comprises:
(i) determining the identity of one or more nucleotides at a polymorphic site in an FGF-BP1 gene of the individual;
(ii) determining the status of the human by reference to said one or more nucleotides; and
(iii) administering an effective amount of the drug.

The polymorphic site will be one which has an association with COPD in a human population. The method preferably comprises determining the nucleotide of the individual at position 184 of SEQ ID NO:1. Preferably, the method comprises determining the presence of base A at a position corresponding to position 184 of SEQ ID NO:1.

Methods of identifying agents (e.g. drugs) capable of modulating FGF-BP1 are described later in this application. Further examples of suitable drugs are antisense molecules (which can be targeted against the mRNA of FGF-BP1) or an antibody or antibody derivative directed against the FGF-BP1 polypeptide or a homologue of the polypeptide. The preparation of antibodies and antisense molecules are described in more detail later in this application.

According to another aspect of the present invention there is provided a method of treating a human having, or at risk of having, COPD, with a drug capable of treating COPD, which method comprises:
(i) determining the identity of one or more nucleotides at a polymorphic site in an FGF-BP1 gene of the individual;
(ii) determining the status of the human by reference to said one or more nucleotides; and
(iii) administering an effective amount of the drug.

The polymorphic site will be one which has an association with COPD in a human population. The method preferably comprises determining the nucleotide of the individual at position 184 of SEQ ID NO:1. Preferably, the method comprises determining the presence of base A at a position corresponding to position 184 of SEQ ID NO: 1.

Examples of drugs which can be used for the treatment of COPD include beta-agonists, and in particular beta-2-agonists (such as formoterol and salmeterol), anticholinergics (such as tiotropium), theophylline, N-acetylcysteine, a combination of a long-acting beta-agonist and an inhaled corticosteroid (such as the combination of formoterol and budesonide, or the combination of fluticasone and salmeterol), or a combination of an anticholinergic and albuterol (such as the combination of albuterol and ipratropium). It should be noted that reference to the above compounds includes pharmaceutically acceptable salts or solvates thereof. For example, the term “formoterol” encompasses the free base as well as e.g. formoterol famarate dehydrate.

According to another aspect of the invention there is provided use of a drug capable of modulating the FGF-BP1 protein in the preparation of a medicament for treating an individual for COPD, wherein the individual has been identified as having a polymorphism which is associated with COPD in the FGF-BP1 gene. Preferably, the polymorphism is a single nucleotide polymorphism. In one embodiment of this aspect of the invention, the polymorphism is at position 184 of SEQ ID NO:1. More preferably, the individual has been identified as having base A at a position corresponding to position 184 of SEQ ID NO:1. The drug or drug combination may be as defined above.

According to another aspect of the invention there is provided use of a drug or drug combination selected from the group consisting of a beta-agonist, an anticholinergic, a combination of a long-acting beta-agonist and an inhaled corticosteroid, a combination of an anticholinergic and albuterol, theophylline and N-acetylcysteine, in the preparation of a medicament for treating an individual for COPD, wherein the individual has been identified as having a polymorphism which is associated with COPD in the FGF-BP1 gene. Preferably, the polymorphism is a single nucleotide polymorphism. In one embodiment, the polymorphism is at position 184 of SEQ ID NO:1 as defined above.

As mentioned above, the present invention identifies for the first time an association of the FGF-BP1 gene with the respiratory disease COPD. The present invention therefore identifies a functional role for FGF-BP1.

Accordingly, in another aspect of the invention there is provided a method for treating COPD in an individual, which method comprises modulating FGF-BP1 expression or functional activity.

The present invention also provides an assay for identifying a compound as a potential treatment of COPD, which assay comprises determining that the compound modulates the function or expression of the FGF-BP1 polypeptide or a homologue thereof or a fragment of either.

By FGF-BP1 is meant a polypeptide corresponding to the amino acid sequence shown in SEQ ID NO:2. The term also encompasses the polypeptide encoded by the sequence set out in SEQ ID NO:1.

It is understood that the polypeptide for use in the invention may be both a fragment and a homologue of the FGF-BP1 polypeptide.

The term “fragment” as used herein refers to a subsequence of the full length sequence that comprises at least 25, preferably at least 50, more preferably at least 100 consecutive amino acids of the sequence depicted in SEQ ID NO: 2. Preferably the fragment is a polypeptide that is the FGF-BP1 protein with either or both C-terminal and N-terminal truncations.

In a preferred embodiment, the screening methods of the invention are carried out using a polypeptide comprising an amino acid sequence as depicted in SEQ ID NO: 2, or a sequence possessing, in increasing order of preference, at least 80%, 85%, 90%, 95%, 97%, 98% and 99% amino acid sequence identity thereto. Such variants are herein referred to as “homologues”.

The sequence identity between two sequences can be determined by pair-wise computer alignment analysis, using programs such as, BestFit, Gap or FrameAlign. The preferred alignment tool is BestFit. In practice, when searching for similar/identical sequences to the query search, from within a sequence database, it is generally necessary to perform an initial identification of similar sequences using suitable software such as Blast, Blast2, NCBI Blast2, WashU Blast2, FastA, Fasta3 and PILEUP, and a scoring matrix such as Blosum 62. Such software packages endeavor to closely approximate the “gold-standard” alignment algorithm of Smith-Waterman. Thus, the preferred software/search engine program for use in assessing similarity, i.e. how two primary polypeptide sequences line up, is Smith-Waterman. Identity refers to direct matches, similarity allows for conservative substitutions.

Any compound identified in this way may prove useful in the treatment of COPD in humans and/or other animals.

Accordingly, in one embodiment, the method for treating COPD comprises treating the individual so as to modulate the function or expression of the FGF-BP1 polypeptide.

As defined herein, modulation includes any effect on the functional activity of FGF-BP1 or the expression thereof. Thus modulation may include, for example, any one or more of the following: conformational change, covalent modification, or inhibition. Modulators include inhibitors (such as antagonists). Modulation of FGF-BP1 protein by a compound may be brought about, for example, through compound binding to the FGF-BP1 protein. The term modulates and modulating should be construed accordingly.

Whether a given agent acts as an FGF-BP1 modulator can be determined, for example, by the following methods:

by functional assays of the FGF-BP1 polypeptide, to determine whether its activity is modulated;
by direct measurement of the binding or interaction of the compound with FGF-BP1 (including competitive binding assays);
by immunological assays (for example, using an antibody specific for FGF-BP1 to determine whether protein levels of FGF-BP1 are affected);
by assays to determine whether gene expression of FGF-BP1 is affected.

Methods for modulating FGF-BP1 function include introducing a compound which interacts with the FGF-BP1 or a homologue thereof or a fragment of either. Such compounds can be identified in screening assays.

In another aspect of the invention there is provided an assay for screening for a compound that modulates the function of FGF-BP1 polypeptide, and which can be used for the treatment of COPD.

Thus, the present invention also provides a method of screening for a compound which can be used to treat COPD, which method comprises:

(a) providing a sample containing FGF-BP1 polypeptide or a homologue thereof or a fragment of either, and a candidate compound; and
(b) detecting the binding of the FGF-BP1 polypeptide, homologue or fragment, to the candidate compound in the sample.

In an embodiment of this aspect, the method comprises:

(a) providing a sample containing FGF-BP1 polypeptide or a homologue thereof or a fragment of either, and a candidate compound; and
(b) measuring the binding of the FGF-BP1 polypeptide, homologue or fragment to the candidate compound in the sample; and
comparing the binding of FGF-BP1 polypeptide, homologue or fragment to the candidate compound in the sample with the binding of the FGF-BP1 polypeptide, homologue or fragment to a control agent, wherein the control agent is known to not bind to the FGF-BP1 polypeptide;
wherein an increase in the binding of the FGF-BP1 polypeptide, homologue or fragment to the candidate compound in the sample relative to the binding of the FGF-BP1 polypeptide, homologue or fragment to the control agent indicates that the candidate compound modulates the function of FGF-BP1 polypeptide.

The functional activity of FGF-BP1 may be modified by suitable compounds (molecules/agents) which bind either directly or indirectly to FGF-BP1 protein, or to the nucleic acid encoding it. Compounds may be naturally occurring molecules such as peptides and proteins, for example antibodies, or they may be synthetic molecules. Methods of modulating the level of expression of FGF-BP1 include, for example, using antisense techniques. Antisense constructs are described in detail in U.S. Pat. No. 6,100,090 (Monia et al), and Neckers et al., 1992, Crit. Rev Oncog 3(1-2):175-231, the teachings of which document are specifically incorporated by reference.

Thus, according to another aspect of the invention, the FGF-BP1 gene may be used in gene therapy, for example where it is desired to modify the production of the protein in vivo, and the invention extends to such uses.

Knowledge of the gene according to the invention also provides the ability to regulate its expression in vivo by for example the use of antisense DNA or RNA. One therapeutic means of inhibiting or dampening the expression levels of a particular gene (for example FGF-BP1 identified herein) is to use antisense therapy. Antisense therapy utilises antisense nucleic acid molecules that are synthetic segments of DNA or RNA (“oligonucleotides”), designed to mirror specific mRNA sequences and block protein production. Once formed, the mRNA binds to a ribosome, the cell's protein production “factory” which effectively reads the RNA sequence and manufactures the specific protein molecule dictated by the gene. If an antisense molecule is delivered to the cell (for example as native oligonucleotide or via a suitable antisense expression vector), it binds to the messenger RNA because its sequence is designed to be a complement of the target sequence of bases. Once the two strands bind, the mRNA can no longer dictate the manufacture of the encoded protein by the ribosome and is rapidly broken down by the cell's enzymes, thereby freeing the antisense oligonucleotide to seek and disable another identical messenger strand of mRNA.

Thus, according to another aspect of the invention there is provided a method for treating a patient suffering from COPD comprising administering to said patient an effective amount of an anti-sense molecule capable of binding to the mRNA of the FGF-BP1 gene, and inhibiting expression of the protein product of the FGF-BP1 gene.

Complete inhibition of protein production is not essential, indeed may be detrimental. It is likely that inhibition to a state similar to that in normal tissues would be desired.

This aspect of antisense therapy is particularly applicable if the COPD disorder is a direct cause of over-expression of the FGF-BP1 gene in question, although it is equally applicable if said FGF-BP1 gene indirectly cause the COPD. With knowledge of the FGF-BP1 gene and mRNA sequence, the person skilled in the art is able to design suitable antisense nucleic acid therapeutic molecules and administer them as required.

Antisense oligonucleotide molecules with therapeutic potential can be determined experimentally using well established techniques. To enable methods of down-regulating expression of the FGF-BP1 gene of the present invention in mammalian cells, an example antisense expression construct can be readily constructed for instance using the pREP10 vector (Invitrogen Corporation). Transcripts are expected to inhibit translation of the gene in cells transfected with this type of construct. Antisense transcripts are effective for inhibiting translation of the native gene transcript, and capable of inducing the effects (e.g., regulation of tissue physiology) herein described. Oligonucleotides which are complementary to and hybridisable with any portion of FGF-BP1 gene mRNA are contemplated for therapeutic use. U.S. Pat. No. 5,639,595, “Identification of Novel Drugs and Reagents”, issued Jun. 17, 1997, wherein methods of identifying oligonucleotide sequences that display in vivo activity are thoroughly described, is herein incorporated by reference. Expression vectors containing random oligonucleotide sequences derived from the FGF-BP1 gene sequence are transformed into cells. The cells are then assayed for a phenotype resulting from the desired activity of the oligonucleotide. Once cells with the desired phenotype have been identified, the sequence of the oligonucleotide having the desired activity can be identified. Identification may be accomplished by recovering the vector or by polymerase chain reaction (PCR) amplification and sequencing the region containing the inserted nucleic acid material. Antisense molecules can be synthesised for antisense therapy. These antisense molecules may be DNA, stable derivatives of DNA such as phosphorothioates or methylphosphonates, RNA, stable derivatives of RNA such as 2′-O-alkylRNA, or other oligonucleotide mimetics. U.S. Pat. No. 5,652,355, “Hybrid Oligonucleotide Phosphorothioates”, issued Jul. 29, 1997, and U.S. Pat. No. 5,652,356, “Inverted Chimeric and Hybrid Oligonucleotides”, issued Jul. 29, 1997, which describe the synthesis and effect of physiologically-stable antisense molecules, are incorporated by reference. Antisense molecules may be introduced into cells by microinjection, liposome encapsulation or by expression from vectors harboring the antisense sequence.

As noted above, antisense nucleic acid molecules may also be provided as RNAs, as some stable forms of RNA are now known in the art with a long half-life that may be administered directly, without the use of a vector. In addition, DNA constructs may be delivered to cells by liposomes, receptor mediated transfection and other methods known to the art.

The antisense DNA or RNA for co-operation with the gene contained in SEQ ID No:1 can be produced using conventional means, by standard molecular biology and/or by chemical synthesis as described above. If desired, the antisense DNA or antisense RNA may be chemically modified so as to prevent degradation in vivo or to facilitate passage through a cell membrane and/or a substance capable of inactivating mRNA, for example ribozyme, may be linked thereto and the invention extends to such constructs.

The antisense DNA or antisense RNA may be of use in the treatment of COPD in humans in which the over- or under-regulated production of the FGF-BP1 gene product has been implicated.

Thus, in a further aspect of the invention there is provided a method of treatment of a patient suffering from COPD, comprising administration to the patient of a compound capable of reducing the transcription or expression of FGF-BP1.

Also provided is a method of treatment of a patient suffering from COPD, comprising administration to the patient an antisense nucleic acid molecule targeted against the mRNA of FGF-BP1.

The invention also provides the use of an antisense nucleic acid molecule directed against FGF-BP1, in the manufacture of a medicament for treating a COPD disorder.

Other methods of modulating gene expression are known to those skilled in the art and include dominant negative approaches as well as introducing peptides or small molecules which inhibit gene expression or functional activity.

In addition, changes in events immediately down-stream of FGF-BP1 activity, such as the modulation of intracellular messengers or expression of genes whose transcription is regulated by FGF-BP1 expression, can be used as an indication that a molecule in question affects the functional activity of FGF-BP1.

The FGF-BP1 protein of the invention and homologues or fragments thereof may be used to generate substances which selectively bind to it and in so doing regulate the activity of the protein and hence COPD. Such substances include, for example, antibodies, and the invention extends in particular to an antibody which is capable of binding to the FGF-BP1 polypeptide. In particular the antibody may be a neutralising antibody.

As used herein the term antibody is to be understood to mean a whole antibody or a fragment thereof, for example a F(ab)₂, Fab, FV, VH or VK fragment, a single chain antibody, a multimeric monospecific antibody or fragment thereof, or a bi- or multi-specific antibody or fragment thereof. Each of these types of antibody derivative and their acronyms are well known to the person skilled in the art.

In another preferred embodiment antibodies directed against FGF-BP1 protein can be used, to detect, prognose, diagnose and stage COPD. Various histological staining methods known in the art, including immunochemical staining methods, may also be used. Silver stain is but one method of detecting FGF-BP1 proteins. For other staining methods useful in the present invention see, for example, A Textbook of Histology, Eds. Bloom and Fawcett, W.B. Saunders Co., Philadelphia (1964).

According to a further aspect of the invention there is provided use of an antibody selective for FGF-BP1 protein, in an assay to diagnose or prognose or monitor COPD.

Methods of making and detecting labelled antibodies are well known (Campbell; Monoclonal Antibody Technology, in: Laboratory Techniques in Biochemistry and Molecular Biology, Volume 13. Eds: Burdon R et al. Elsevier, Amsterdam (1984)). The term antibody includes both monoclonal antibodies, which are a substantially homogeneous population, and polyclonal antibodies which are heterogeneous populations. The term also includes inter alia, humanised and chimeric antibodies. Monoclonal antibodies to specific antigens may be obtained by methods known to those skilled in the art, such as from hybridoma cells, phage display libraries or other methods. Monoclonal antibodies may be inter alia, human, rat or mouse derived. For the production of human monoclonal antibodies, hybridoma cells may be prepared by fusing spleen cells from an immunised animal, e.g. a mouse, with a tumour cell. Appropriately secreting hybridoma cells may thereafter be selected (Koehler & Milstein, Nature 256:495-497 (1975); Cole et al., “Monoclonal antibodies and Cancer Therapy”, Alan R Liss Inc, New York N.Y. pp 77-96 (1985)). Such antibodies may be of any immunoglobulin class including IgG, IgM, IgE, IgA, IgD and any subclass thereof.

Polyclonal antibodies can be generated by immunisation of an animal (such as a mouse, rat, goat, horse, sheep etc) with an antigen, such as one of the FGF-BP1 proteins used in this invention.

The FGF-BP1 polypeptide (or homologues/variants thereof) can be prepared by various techniques known to the person skilled in the art. RNA transcripts can be used to prepare a polypeptide of the invention by in vitro translation techniques according to known methods (Sambrook et al. supra). Alternatively, FGF-BP1 can be synthesised chemically. For example, by the Merryfield technique (J. Amer. Chem. Soc. 85:2149-2154, (1968)). Numerous automated polypeptide synthesisers, such as Applied Biosystems 431A Peptide Synthesizer also now exist. Alternatively, and preferably, the FGF-BP1 polypeptide is produced from a nucleotide sequence encoding the polypeptide using recombinant expression technology. A variety of expression vector/host systems may be used to express the FGF-BP1 coding sequences. These include, but are not limited to microorganisms such as bacteria transformed with plasmids, cosmids or bacteriophage; yeasts transformed with expression vectors; insect cell systems transfected with baculovirus expression systems; plant cell systems transfected with plant virus expression systems, such as cauliflower mosaic virus; or mammalian cell systems (for example those transfected with adenoviral vectors); selection of the most appropriate system is a matter of choice. Preferably, the FGF-BP1 protein is expressed in eukaryotic cells, especially mammalian, insect and yeast cells. Mammalian cells provide post-translational modifications to recombinant FGF-BP1 protein, which include folding and/or phosphorylation.

Expression vectors usually include an origin of replication, a promoter, a translation initiation site, optionally a signal peptide, a polyadenylation site, and a transcription termination site. These vectors also usually contain one or more antibiotic resistance marker gene(s) for selection. As noted above, suitable expression vectors may be plasmids, cosmids or viruses such as phage or retroviruses. The coding sequence of the polypeptide is placed under the control of an appropriate promoter, control elements and transcription terminator so that the nucleic acid sequence encoding the polypeptide is transcribed into RNA in the host cell transformed or transfected by the expression vector construct. The coding sequence may or may not contain a signal peptide or leader sequence for secretion of the polypeptide out of the host cell. Expression and purification of the FGF-BP1 polypeptide can be easily performed using methods well known in the art (for example as described in Sambrook et al. supra).

The FGF-BP1 polypeptide so produced can then be used to inoculate animals, from which serum samples, containing the specific antibody against the introduced FGF-BP1 protein/polypeptide, can later be obtained.

Rodent antibodies may be humanised using recombinant DNA technology according to techniques known in the art. Alternatively, chimeric antibodies, single chain antibodies, Fab fragments may also be developed against the polypeptides of the invention (Huse et al., Science 256:1275-1281 (1989)), using skills known in the art. Antibodies so produced have a number of uses which will be evident to the molecular biologist or immunologist skilled in the art. Such uses include, but are not limited to, monitoring enzyme expression, development of assays to measure enzyme activity and use as a therapeutic agent. Enzyme linked immunosorbant assays (ELISAs) are well known in the art and would be particularly suitable for detecting the FGF-BP1 protein or polypeptide fragments thereof in a test sample.

The FGF-BP1 specific antibodies can be used in an ELISA assay to detect FGF-BP1 protein in body fluids or by immunohistochemistry or other means. In addition, an antibody could be used individually or as part of a panel of antibodies, together with a control antibody which reacts to a common protein, on a dipstick or similar diagnostic device.

The present invention also provides a method of screening compounds to identify antagonists to FGF-BP1 or variants of FGF-BP1. Candidate compounds may be identified from a variety of sources, for example, cells, cell-free preparations, chemical libraries, peptide and gene libraries, and natural product mixtures. Chemical libraries include combinatorial chemistry libraries and, in particular, a combinatorial chemical library comprising compounds that interact with GPCRs. Such antagonists or inhibitors so-identified may be natural or modified substrates, ligands, receptors, enzymes, antibodies (as described above) etc., as the case may be, of the FGF-BP1 protein; or may be structural or functional mimetics thereof (see Coligan et al., Current Protocols in Immunology 1(2):Chapter 5 (1991)).

Techniques such as analytical centrifugation, affinity binding studies involving chromatography or electrophoresis can be used to detect molecules which interact directly with FGF-BP1. Other techniques that allow the identification of protein-protein interactions include immunoprecipitation and yeast two hybrid studies.

Compounds having inhibitory, activating, or modulating activity can be identified using in vitro and in vivo assays for FGF-BP1 activity and/or expression, e.g., ligands, agonists, antagonists, and their homologs and mimetics.

The screening methods of the present invention may simply measure the binding of a candidate compound (agent) to the FGF-BP1, or to cells or membranes bearing FGF-BP1, or a fusion protein thereof by means of a label directly or indirectly associated with the candidate compound. Alternatively, the screening method may involve competition with a labelled competitor. Further, these screening methods may test whether the candidate compound results in a signal generated by activation or inhibition of FGF-BP1, using detection systems appropriate to the cells bearing the receptor.

For example, a cell or membrane preparation expressing FGF-BP1 may be contacted with a compound of interest. The ability of the compound to generate (or suppress) a response, following interaction with FGF-BP1 is then measured. A parallel sample which does not receive the test compound is also monitored as a control. The treated and untreated cells or membranes are then compared by any suitable phenotypic criteria, including but not limited to microscopic analysis, chemotaxis, viability testing, ability to replicate, histological examination, the level of a particular RNA or polypeptide associated with the cells, the level of enzymatic activity expressed by the cells or cell lysates, and the ability of the cells to interact with other cells or compounds. Such methods are known in the art (eg Neote K, et al., Cell, 72:415-25 (1993)).

A compound which binds but does not elicit a response identifies that compound as an antagonist. An antagonist compound is also one which binds and produces an opposite response. A compound which binds and elicits a response is identified as an agonist.

Inhibitors of FGF-BP1 activation may be assayed in the presence of a known agonist and the effect on activation by the agonist by the presence of the candidate compound is observed. Constitutively active polypeptides may be employed in screening methods for inverse agonists or inhibitors, in the absence of an agonist or inhibitor, by testing whether the candidate compound results in inhibition of activity of FGF-BP1. Further, the screening methods may simply comprise the steps of mixing a candidate compound with a solution containing FGF-BP1, to form a mixture, and determining whether its ability to bind FGF-BP1 protein is reduced. Fusion proteins, such as those made from Fc portion and FGF-BP1, may also be used for high-throughput screening assays to identify antagonists for, FGF-BP1 function (see D. Bennett et al., J Mol Recognition, 8:52-58 (1995); and K. Johanson et al., J. Biol. Chem., 270(16):9459-9471 (1995)).

According to a further aspect of the invention there is provided a screening assay or method for identifying potential anti-COPD disorder therapeutic compounds comprising contacting an assay system capable of detecting the effect of a test compound against expression level of FGF-BP1, with a test compound and assessing the change in expression level of FGF-BP1.

Compounds that modulate the expression of DNA or RNA of FGF-BP1 polypeptides may be detected by a variety of assay systems. A suitable assay system may be a simple “yes/no” assay to determine whether there is a change in expression of a reporter gene, such as beta-galactosidase, luciferase, green fluorescent protein or others known to the person skilled in the art (reviewed by Naylor, Biochem. Pharmacol. 58:749-57 (1999)). The assay system may be made quantitative by comparing the expression or function of a test sample with the levels of expression or function in a standard sample. Systems in which transcription factors are used to stimulate a positive output, such as transcription of a reporter gene, are generally referred to as “one-hybrid systems” (Wang, M. M. and Reed, R. R. (1993) Nature 364:121-126). Using a transcription factor to stimulate a negative output (growth inhibition) may thus be referred to as a “reverse one-hybrid system” (Vidal et al, 1996, supra). Therefore, in an embodiment of the present invention, a reporter gene is placed under the control of the FGF-BP1 promoter.

According to a further aspect of the invention there is provided a method of testing potential therapeutic agents for the ability to suppress COPD comprising contacting a test compound with a cell engineered to express the FGF-BP1 polypeptide; and determining whether said test compound suppressed expression of the FGF-BP1 polypeptide.

Also provided is a method for identifying inhibitors of transcription of FGF-BP1, which method comprises contacting a potential therapeutic agent with a cell or cell line as described above and determining inhibition of FGF-BP1 transcription by the potential therapeutic agent by reference to a lack of or reduction in expression of the reporter gene.

As described above, any convenient test compound or library of test compounds may be used in conjunction with the test assay. Particular test compounds include low molecular weight chemical compounds (preferably with a molecular weight less than 1500 daltons) suitable as pharmaceutical or veterinary agents for human or animal use, or compounds for non-administered use such as cleaning/sterilising agents or for agricultural use. Test compounds may also be biological in nature, such as antibodies (see above).

According to a further aspect of the invention there is provided a compound identified by a screening method as defined herein.

According to a further aspect of the invention, there is provided a method of treatment of a patient suffering from COPD comprising administration to said patient of an effective amount of a compound identified according to a screening method of the invention.

It will be appreciated that the term “for the treatment of COPD”, and variations thereon, includes therapeutic and prophylactic (preventative) treatment.

According to another aspect of the present invention there is provided a method of preparing a pharmaceutical composition which comprises:

i) identifying a compound as useful for treatment of COPD according to a screening method as described herein; and
ii) mixing the compound or a pharmaceutically acceptable salt thereof with a pharmaceutically acceptable excipient or diluent.

According to a further aspect of the invention there is provided a method of treatment of a patient suffering from COPD comprising administration to said patient of an effective amount of a compound identified according to a screening method of the invention or a composition prepared by the method described herein.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal track, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate), anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an ester or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedure well known in the art.

Compositions for administration by insufflation may be in the form of a finely divided powder containing particles of average diameter of, for example, 30μ or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on Formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The size of the dose for therapeutic or prophylactic purposes of a compound will naturally vary according to the nature and severity of the conditions, the age and sex of the animal or patient and the route of administration, according to well known principles of medicine.

In using a compound for therapeutic or prophylactic purposes it will generally be administered so that a daily dose in the range, for example, 0.5 mg to 75 mg per kg body weight is received, given if required in divided doses. In general lower doses will be administered when a parenteral route is employed. Thus, for example, for intravenous administration, a dose in the range, for example, 0.5 mg to 30 mg per kg body weight will generally be used. Similarly, for administration by inhalation, a dose in the range, for example, 0.5 mg to 25 mg per kg body weight will be used. Oral administration is however preferred.

As mentioned above, having identified that the FGF-BP1 gene is implicated in COPD, this presents many molecular diagnostic opportunities. In this connection, it is known to persons skilled in the art that clinically significant information may be obtained by the measurement of the levels of nucleic acids, proteins or other analytes that occur within biological samples.

An investigator may wish to measure the levels of FGF-BP1 protein or to measure the levels of FGF-BP1 mRNA transcript present in a sample. An investigator may also wish to perform nucleic acid sequence analyses to detect further variant nucleotides (SNPs) present within the sample, these analyses may be performed on either the DNA or RNA fraction of the sample and are well known to the person skilled in the art. An investigator may also wish to perform protein sequence analysis either directly by degradation based techniques which are well known in the art or indirectly by molecular recognition techniques including immunoassay or by techniques based on detecting changes in the physical characteristics of the protein such as functional or substrate specificity assays or iso-electric focusing.

According to a further aspect of the invention there is provided a method for diagnosing or prognosing or monitoring COPD, comprising testing a biological sample for aberrant levels of FGF-BP1. Preferably, the sample has already been removed from the body of an individual.

The term “aberrant levels” refers to levels that are outside the normal range. The normal range can be determined by testing many normal tissues or may be determined from a side by side comparison of the test sample with the normal or control sample. For the purposes of this application, aberrant expression refers to a 1.5-fold difference or more in level of nucleic acid in a disease sample compared to control normal. Nucleic acid as used herein refers to both RNA and DNA.

In principle, any diagnostic method capable of assessing the differential expression level, relative to expression in control tissues, of the FGF-BP1 gene identified herein, can be used, either alone or as a panel. In particular, such methods include assessment of mRNA transcript levels and/or protein levels. The presence of aberrant expression levels of the gene may indicate the presence of COPD or an increased likelihood to develop the disorder.

The amino acid sequence method for diagnosis may be one which is determined by immunological methods such as enzyme linked immunosorbent assay (ELISA).

The levels of the FGF-BP1 can be assessed from relative amounts of mRNA, cDNA, genomic DNA or polypeptide sequence present in the test sample. Where RNA is used, it may be desired to convert the RNA to a complementary cDNA and during this process it may be desirable to incorporate a suitable detectable label into the cDNA.

In a particular embodiment the method of the invention relies on detection of mRNA transcript levels. This involves assessment of the relative mRNA transcript levels of FGF-BP1 in a sample, and comparison of sample data to control data. The gene transcript can be detected individually, or, is preferably detected amongst a panel of other disease-linked genes from which a transcript profile can be generated. Levels of FGF-BP1 mRNA in the test sample can be detected by any technique known in the art. These include Northern blot analysis, reverse transcriptase-PCR amplification (RT-PCR), microarray analysis and RNAse protection.

In one embodiment, levels of FGF-BP1 RNA in a sample can be measured in a Northern blot assay. Here, tissue RNA is fractionated by electrophoresis, fixed to a solid membrane support, such as nitrocellulose or nylon, and hybridised to a probe or probes capable of selectively hybridising with the FGF-BP1 RNA to be detected. The actual levels may be quantitated by reference to one or more control housekeeping genes. Probes may be used singly or in combination. This may also provide information on the size of mRNA detected by the probe. Housekeeping genes are genes which are involved in the general metabolism or maintenance of the cell, and are considered to be expressed at a constant level irrespective of cell type, physiological state or stage in the cell cycle. Examples of suitable housekeeping genes are: beta actin, GAPDH, histone H3.3 or ribosomal protein L13 (Koehler et al., Quantitation of mRNA by Polymerase Chain Reaction. Springer-Verlag, Germany (1995)).

To gauge relative expression levels, a control sample can be run alongside the test sample or, the test result/value can be compared to FGF-BP1 expression levels expected in a normal or control tissue. These control values can be generated from prior test experiments using normal or control tissues, to generate mean or normal range values for FGF-BP1.

In another embodiment, the FGF-BP1 nucleic acid in a tissue sample is amplified and quantitatively assayed. The polymerase chain reaction (PCR) procedure can be used to amplify specific nucleic acid sequences through a series of iterative steps including denaturation, annealing of oligonucleotide primers (e.g., designed according to the sequence disclosed in SEQ ID NO. 1), and extension of the primers with DNA polymerase (see, for example, Mullis, et al., U.S. Pat. No. 4,683,202; Loh et al., Science 243:217 (1988)). In reverse transcriptase-PCR(RT-PCR) this procedure is preceded by a reverse transcription step to allow a large amplification of the number of copies of mRNA (Koehler et al., supra). Other known nucleic acid amplification procedures include transcription-based amplification systems (TAS) such as nucleic acid based sequence application (NASBA) and 3SR (Kwoh et al., Proc Natl. Acad Sci USA 86:1173 (1989), Gingeras et al., PCT application WO 88/10315), the ligase chain reaction (LCR, see European Application No. 320308), Strand Displacement Amplification (SDA), “race”, “one sided PCR” and others (Frohman, PCR Protocols: a Guide to Methods and Applications. Academic Press, NY (1990); Ohara et al., Proc Natl Acad. Sci. USA 86:5673-5677 (1989)). Quantitation of RT-PCR products can be done while the reaction products are building up exponentially, and can generate diagnostically useful clinical data. In one embodiment, analysis is carried out by reference to one or more housekeeping genes which are also amplified by RT-PCR. Quantitation of RT-PCR product may be undertaken, for example, by gel electrophoresis visual inspection or image analysis, HPLC (Koehler et al., supra) or by use of fluorescent detection methods such as intercalation labelling, Taqman probe (Higuchi et al., Biotechnology 10:413-417 (1992)), Molecular Beacon (Piatek et al., Nature Biotechnol. 4:359-363 (1998)), primer or Scorpion primer (Whitcombe et al., Nature Biotech 17:804-807 (1999)); or other fluorescence detection method, relative to a control housekeeping gene or genes as discussed above.

FGF-BP1 RNA measurements can also be carried out on sinovial fluid, blood or serum samples. Preferably, the RNA is obtained from a peripheral blood sample. In the case of soluble RNA in the blood serum, the low abundance of mRNA expected would necessitate a sensitive test such as RT-PCR (Kopreski et al., Clin Cancer Res 5:1961-5 (1999)). A whole blood gradient may be performed to isolate nucleated cells and total RNA is extracted such as by the Rnazole B method (Tel-Test Inc., Friendsworth, Tex.) or by modification of methods known in the art such as described in Sambrook et al., (supra).

In a particular embodiment of the invention, the diagnosis/detection method of the invention involves assessing FGF-BP1 transcript levels using microarray analysis. Microarray technology makes it possible to simultaneously study the expression of many thousands of genes in a single experiment. Analysis of gene expression in human tissue (e.g. biopsy tissue) can assist in the diagnosis and prognosis of disease and the evaluation of risk for disease. A comparison of levels of expression of various genes from patients with defined pathological disease conditions with normal patients enables an expression profile, characteristic of disease, to be created.

Probes are made that selectively hybridise to the sequences of the target FGF-BP1 gene in the test sample. These probes, perhaps together with other probes and control probes, are bound at discrete locations on a suitable support medium such as a nylon filter or microscope slide to form a transcript profiling array. The diagnostic method involves assessing the relative mRNA transcript level of the FGF-BP1 in a clinical sample. This can be done by radioactively labelling, or non-radioactively labelling the tissue mRNA, which can be optionally purified from total RNA, in any of a number of ways well known to the art (Sambrook et al., supra). The probes can be directed to any part or all of the target FGF-BP1 mRNA.

In another embodiment of the invention, total FGF-BP1 RNA or DNA is quantified and compared to levels in control tissue or expected levels from pre-tested standards. DNA and/or RNA may be quantified using techniques well known in the art. Messenger RNA is often quantitated by reference to internal control mRNA levels within the sample, often relative to housekeeping genes (Koehler et al., supra).

In a further embodiment hybridisation signals generated are measured by computer software analysis of images on phosphorimage screens exposed to radioactively labelled tissue RNA hybridised to a microarray of probes on a solid support such as a nylon membrane. In another, quantities are measured by densitometry measurements of radiation-sensitive film (e.g. X-ray film), or estimated by visual means. In another embodiment quantities are measured by use of fluorescently labelled probe, which may be a mixture of biopsy and normal RNA differentially labelled with different fluorophores, allowing quantities of FGF-BP1 mRNA to be expressed as a ratio versus the normal level. The solid support in this type of experiment is generally a glass microscope slide, and detection is by fluorescence microscopy and computer imaging.

The detection of specific interactions may be performed by detecting the positions where the labelled target sequences are attached to the array. Radiolabelled probes can be detected using conventional autoradiography techniques. Use of scanning autoradiography with a digitised scanner and suitable software for analysing the results is preferred. Where the label is a fluorescent label, the apparatus described, e.g. in International Publication No. WO 90/15070; U.S. Pat. No. 5,143,854 or U.S. Pat. No. 5,744,305 may be advantageously applied. Indeed, most array formats use fluorescent readouts to detect labelled capture:target duplex formation. Laser confocal fluorescence microscopy is another technique routinely in use (Kozal et al., Nature Medicine 2:753-759 (1996)). Mass spectrometry may also be used to detect oligonucleotides bound to a DNA array (Little et al, Analytical Chemistry 69: 4540-4546, (1997)). Whatever the reporter system used, sophisticated gadgetry and software may be required in order to interpret large numbers of readouts into meaningful data (such as described, for example, in U.S. Pat. No. 5,800,992 or International Publication No. WO 90/04652).

In a particular embodiment of the microarray test, the FGF-BP1 RNA measurement is generated as a value relative to an internal standard (i.e. a housekeeping gene) known to be constant or relatively constant. The histone H3.3 and ribosomal protein L19 housekeeping genes have been shown to be cell-cycle independent and constitutively expressed in all tissues (Koehler et al., supra). For normalisation of data, several different housekeeping genes can be used to generate an average housekeeping measurement.

A microarray or RT-PCR test to detect COPD disorder or susceptibility thereto can be used where tissue samples containing mRNA are available.

Samples for RNA extraction must be treated promptly to avoid RNA degradation (Sambrook et al., supra). This entails either prompt extraction using e.g. phenol-based reagents or snap freezing in e.g. liquid Nitrogen. Samples can be stored at −70° C. or less until RNA can be extracted at a later date. Proprietary reagents are available which allow tissue or cells to be conveniently stored for several days at room temperature and up to several months at 4° C. (e.g. RNAlater, Ambion Inc., TX). Prior to extraction, methods such as grinding, blending or homogenisation are used to dissipate the tissue in a suitable extraction buffer. Typical protocols then use solvent extraction and selective precipitation techniques.

In another embodiment, oligonucleotide probe(s) capable of selectively hybridising to FGF-BP1 nucleic acid, can be used to detect levels of FGF-BP1 gene expression.

Convenient DNA sequences for use in the various aspects of the invention may be obtained using conventional molecular biology procedures, for example by probing a human genomic or cDNA library with one or more labelled oligonucleotide probes containing 10 or more contiguous nucleotides designed using the nucleotide sequences described here. Alternatively, pairs of oligonucleotides one of which is homologous to the sense strand and one to the antisense strand, designed using the nucleotide sequences described here to flank a specific region of DNA may be used to amplify that DNA from a cDNA library.

The oligonucleotide primers and probes of the invention (including those used to identify the SNPs identified herein) may be labelled. There are many conventional detectable labels such as radioisotopes, fluorescent labels, chemiluminescent compounds, labelled binding proteins, magnetic labels, spectroscopic markers and linked enzymes that might be used in conjunction with the primers or probes of the invention. One particular example well known in the art is end-labelling with ³²P Fluorescent labels are preferred because they are less hazardous than radiolabels, they provide a strong signal with low background and various different fluorophors capable of absorbing light at different wavelengths and/or giving off different colour signals exist to enable comparative analysis in the same analysis. For example, fluorescein gives off a green colour, rhodamine gives off a red colour and both together give off a yellow colour.

Preferred primers for amplification are between 15 and 60 bp, more preferably between 17 and 35 bp in length. Probe sequences can be anything from about 25 nucleotides in length upwards. If the target sequence is a gene of 2 kb in size the probe sequence can be the complete gene sequence complement and thus may also be 2 kb in size. Preferably, the probe sequence is a genomic, or more preferably a cDNA, fragment of the target sequence and may be between 50 and 2000 bp, preferably between 200 and 750 bp. It will be appreciated that multiple probes each capable of selectively hybridising to a different target sequence of the FGF-BP1 nucleic acid, may be across the complete length of the FGF-BP1 gene sequence, may be prepared and used together in a diagnostic test. The primers or probes may be completely homologous to the target sequence or may contain one or more mismatches to assist specificity in binding to the correct template sequence. Any sequence which is capable of selectively hybridising to the target sequence of interest may be used as a suitable primer or probe sequence. It will also be appreciated that the probe or primer sequences must hybridise to the target template nucleic acid. If the target nucleic acid is double stranded (genomic or cDNA) then the probe or primer sequence can hybridise to the sense or antisense strand. If however the target is mRNA (single stranded sense strand) the primer/probe sequence will have to be the antisense complement.

An example of a suitable hybridisation solution when a nucleic acid is immobilised on a nylon membrane and the probe nucleic acid is greater than 500 bases or base pairs is: 6×SSC (saline sodium citrate), 0.5% SDS (sodium dodecyl sulphate), 100 μg/ml denatured, sonicated salmon sperm DNA. The hybridisation being performed at 68° C. for at least 1 hour and the filters then washed at 68° C. in 1×SSC, or for higher stringency, 0.1×SSC/0.1% SDS.

An example of a suitable hybridisation solution when a nucleic acid is immobilised on a nylon membrane and the probe is an oligonucleotide of between 12 and 50 bases is: 3M trimethylammonium chloride (TMACl), 0.01M sodium phosphate (pH 6.8), 1 mM EDTA (pH 7.6), 0.5% SDS, 100 μg/ml denatured, sonicated salmon sperm DNA and 0.1% dried skimmed milk. The optimal hybridisation temperature (Tm) is usually chosen to be 5° C. below the Ti of the hybrid chain. Ti is the irreversible melting temperature of the hybrid formed between the probe and its target. If there are any mismatches between the probe and the target, the Tm will be lower. As a general guide, the recommended hybridisation temperature for 17-mers in 3M TMACl is 48-50° C.; for 19-mers, it is 55-57° C.; and for 20-mers, it is 58-66° C.

Levels of FGF-BP1 gene expression can also be detected by screening for levels of polypeptide (FGF-BP1 protein). For example, monoclonal antibodies immunoreactive with FGF-BP1 protein can be used to screen a test sample. Such immunological assays can be done in any convenient format known in the art. These include Western blots, immunohistochemical assays and ELISA assays. Functional assays can also be used, such as protein binding determinations.

All the essential materials and reagents required for detecting FGF-BP1 in a test sample may be assembled together in a kit. Such a kit may comprise one or more diagnostic cDNA probes or oligonucleotide primers together with control probes/primers. The kit may contain probes immobilised on a microarray substrate such as a filter membrane or silicon-based substrate. The kit may also comprise samples of total RNA derived from tissues of various physiological states, such as normal, BPH, confined tumour and metastatic tumour, for example, to be used as controls. The kit may also comprise appropriate packaging and instructions for use in the methods of the invention.

According to another aspect of the present invention there is provided a diagnostic kit for diagnosing or prognosing or monitoring COPD comprising, one or more diagnostic probe(s) and/or diagnostic primer(s) and/or antibodies capable of selectively hybridising or binding to FGF-BP1.

It will be appreciated that the term “diagnostic kit” is not intended to limit the kit to diagnostic use only, it also encompasses other uses such as in prognostic, stage monitoring and therapeutic efficacy studies.

In a particular embodiment, the diagnostic (detection) probes are provided on a microarray.

Such kits may further comprise appropriate buffer(s) and/or polymerase(s) such as thermostable polymerases, for example taq polymerase. They may also comprise companion/constant primers and/or control primers or probes. A companion/constant primer is one that is part of the pair of primers used to perform PCR. Such primers usually complement the template strand precisely. The kit may also contain control RNA from normal tissue labelled with one fluorophore (E.g. Cy5). In use, patient RNA derived from biopsy or body fluids or cells can be labelled with another fluorophore (e.g. Cy3), the RNAs could then be mixed and hybridised to the array. Instrumentation to detect fluorescence ratio e.g. of. Cy3:Cy5 are available and could be used to detect COPD by detecting FGF-BP1 over-expression.

In another embodiment the kit comprises one or more specific probes suitable for hybridisation to mRNA in tissue sections in situ. The kit may also contain hybridisation buffer and detection reagents for colourimetric or fluorescence microscopy detection.

In another embodiment the kit comprises a set of specific oligonucleotide primers, optionally labelled, for quantitation by RT-PCR of FGF-BP1 mRNA. These primers may be Scorpion primers (Whitcombe et al., Nature Biotechnol. 17:804-807, 1999) allowing accurate quantitation of specific PCR product. Alternatively, Taqman or Molecular-Beacon probes may be provided in the kit for this purpose. One form of the kit would be a microtitre plate containing specific reagents in several wells, to which aliquots of extracted RNA could be pipetted. The microtitre plate could be thermocycled on a suitable machine, which could also be capable of reading fluorescence emissions from plate wells (e.g. Perkin Elmer 7700).

In another embodiment the kit comprises one or more antibodies specific for the FGF-BP1 protein for use in immunohistochemical analysis.

In another embodiment the kit is an ELISA kit comprising one or more antibodies specific for the FGF-BP1 protein identified herein.

Pathway mapping may be used to determine each protein in the cell with which the COPD protein interacts and, in turn, the proteins with which each of these proteins interacts also. In this way it is possible to identify the specific critical signalling pathway which links the disease stimulus to the cell's response thereby enabling the identification of new potential targets for therapy intervention.

According to a further aspect of the invention there is provided the use of the FGF-BP1 gene or a fragment thereof in research to identify further gene targets implicated in COPD.

According to a further aspect of the invention, there is provided a model animal system for studying COPD by providing an animal having a mutation or knock out for FGF-BP1. Methods for generating suitable transgenic animals are well known to those skilled in the art.

Thus in a further aspect of the invention there is provided a non-human host mammal model for COPD comprising FGF-BP1 gene disrupted.

In one embodiment, the non-human host mammal has a knock out for FGF-BP1.

Recombinant organisms, i.e., genetically modified animals, expressing a FGF-BP1 or a variant FGF-BP1 gene comprising a polymorphism (such as the SNP described herein) can be prepared using standard procedures known in the art. Preferably, a construct comprising FGF-BP1 or a variant thereof is introduced into a non-human animal or an ancestor of the animal at an embryonic stage, i.e., the one-cell stage, or generally not later than about the eight-cell stage. Genetically-modified animals carrying the constructs of the invention can be made by several methods known to those having skill in the art. One method involves transfecting into the embryo a retrovirus constructed to contain a gene or genes of interest, and other components known to those skilled in the art to provide a complete shuttle vector comprising the transgene, see e.g., U.S. Pat. No. 5,610,053. Another method involves directly injecting a transgene into the embryo. A third method involves the use of embryonic stem cells.

Preferably the genetic modification process results in replacement of the animal's FGF-BP1 gene with the human FGF-BP1 gene. Examples of animals into which the human FGF-BP1 isogenes may be introduced include, but are not limited to, mice, rats, other rodents, and nonhuman primates (see “The Introduction of Foreign Genes into Mice” and the cited references therein, In: Recombinant DNA, Eds. J. D. Watson, M. Gilman, J. Witkowski, and M. Zoller; W. H. Freeman and Company, New York, pages 254-272). Recombinant non-human animals stably expressing a human FGF-BP1 isogene and producing human FGF-BP1 protein can be used as biological models for studying diseases related to abnormal FGF-BP1 expression and/or activity, and for screening and assaying various candidate drugs, compounds, and treatment regimens to reduce the symptoms or effects of these diseases. In particular, these non-human animals can be used as models for diseases including COPD.

The present invention will now be described with reference to the following non-limiting examples.

### EXAMPLE 1

### Identification of Polymorphisms In and Around FGF-BP1

A search was made for polymorphisms located within the FGF-BP1 gene, including the 5′ and 3′ flanking regions of the gene, by examining the public SNP databases including the TSC (the SNP consortium), the NCBI (the National Center of Biotechnology Information) and the EBI (European Bioinformatics Institute). These databases have been compiled by aligning DNA sequences from multiple different individuals. Probable polymorphisms are identified as locations of misalignment in otherwise perfectly matched sequences, although they do not accurately reflect allele frequency or even the presence of polymorphisms in the population of interest. The public databases were searched for FGF-BP1 SNPs using the BLAST algorithm to align segments of FGF-BP1 sequence with left and right flanking sequence for each database SNP entry. Genuine alignments were identified by perfect or near perfect matches over >50 bp and the positions of probable SNPs marked on a sequence map of FGF-BP1 which also included FGF-BP1 exons and introns. Segments of the FGF-BP1 gene containing database SNPs were amplified by PCR from 14 individuals, and the amplified products examined by DNA sequencing to confirm or exclude the presence of SNPs. Five database SNPs (rs2245964, rs2531175, rs2245956, rs1042510 and rs732245, located at positions 7762, 7989, 7991, 10151, and 10592 in SEQ ID NO: 1, respectively) and three novel SNPs (located at positions 7446, 9308, 9554, and in SEQ ID NO: 1), not present in the public SNP databases at the time of this work, were confirmed.

The initial polymorphism discovery focussed on the exons, introns and flanking sequences extending up to 2 kb either side of FGF-BP1. To include more distally-located SNPs, Applied Biosystem's Assays on Demand database was searched and an additional SNP was identified (ABI identifier, C_{—}2643069_{—}10; corresponding to position 184 in SEQ ID NO: 1). ABI's database comprises a set of confirmed SNPs plus optimised reagents for high throughput genotyping using the Taqman allelic discrimination assay (see below). Including more distally located SNPs allowed for the possible inclusion of putative FGF-BP1 regulatory regions not covered by our initial screen. The public identifier for this SNP was rs10939644.

### Genetic Association of FGF-BP1 SNPs to COPD

All 9 of the identified SNPs were chosen for genotyping in a sample of approximately 700 patients with COPD and 450 controls matched for pack years of smoking.

Genotyping assays were developed by ABI and then optimised for use in house. For the Assay on Demand SNP, oligonucleotide reagents specific for this SNP, together with a common reaction master mix, were purchased from ABI. Assays on Demand reagents comprise optimised oligonucleotides that specifically detect a single named SNP. For the remaining 8 SNPs, custom reagents were ordered from ABI to detect these SNPs. Working assays were developed for 6 of these SNPs, making 7 in total.

The genotyping methodology employed was the Taqman allelic discrimination assay. PCR primers were chosen to amplify a small segment of nucleic acid containing the SNP of interest. Included in the amplification reactions were two oligonucleotides probes, each one specific to one allele of the SNP. During the course of amplification, each probe hybridised to its target allele, generating fluorescence that was quantitated by a sensitive detector. Since each of the two probes was labelled with a different fluorochrome, usually FAM and VIC, the presence of one or both alleles in patient or control DNA could be determined, and captured electronically. Since each SNP is biallelic, 3 different genotypes are possible, so for a SNP with alleles C and G, the 3 different genotypes are CC, CG and GG. In the Taqman allelic discrimination assay, such a SNP could be genotyped if the C allele were hybridised by a probe labelled with the fluorochrome FAM and the G allele hybridised with a probe labelled with the fluorochrome VIC. In this example, the CC genotype would be characterised by FAM fluorescence only; the CG genotype by FAM and VIC fluorescence; and the GG genotype by VIC fluorescence only. In this way, each of the 7 FGF-BP1 SNPs were genotyped.

TABLE 1
Details of FGF-BP1 SNPs for which
genotyping assays were developed.

| | | Base change | |
|---|---|---|---|
| Mutdb | Position in | (common | Location in |
| identifier | SEQ ID NO: 1 | allele first) | FGF-BP1 gene |
| rs10939644 | 184 | G/A | 5′ flank |
| rs12503796 | 7446 | G/T | intron 1 |
| rs2245964 | 7762 | G/C | exon 2 (5′UTR) |
| rs2531175 | 7989 | T/C | intron 2 |
| rs2245956 | 7991 | A/C | intron 2 |
| rs1042510 | 10151 | C/T | exon 3 (3′UTR) |
| rs732245 | 10592 | G/A | 3′ flank |

The COPD patients and controls were ascertained from clinical trial centres in Belgium, Denmark, Finland, Italy, The Netherlands, Norway, Spain, Sweden and the UK. A diagnosis of COPD was confirmed by respiratory physicians and all patients (cases) had mild disease. The matching of cases and controls for geographical location as well as smoking increases the utility of this DNA collection for genetic studies by reducing the chance of a false positive result related to population substructure, and controlling for known environmental risk factors for COPD. The DNA collection is also appropriately sized to detect a genetic effect due to a susceptibility allele, say of genetic risk 2.0. Although the number and strength of susceptibility alleles for COPD is not known, it is reasonable to speculate that there are multiple alleles with genetic risks in the 1.2-2.0 range.

The genotyping data for each of the SNPs was analysed for genetic association to COPD in two ways. In the first, the number of individuals with each of the three possible genotypes for each SNP was compared between cases and controls. Chi-squared tests were performed which compared the observed distributions with those expected if there was no association. This test identifies genetic associations related to specific genotypes, such as that caused by dominance of one allele over the other, in the absence of allele frequency differences between cases and controls. For each SNP, a p value was generated which is the probability of the observed result due to chance. A p value equal to, or less than, 0.05 was taken as evidence of genetic association between a single SNP and COPD.

The second analysis method compares the allele frequencies for each SNP between cases and controls. This allele-wise method is not sensitive to deviations in Hardy-Weinberg equilibrium like that seen with the genotype-based method described above and is most suitably applied when there is no evidence of dominance. In the allele-wise method, odds ratios are calculated for each SNP, which are a measure of the disease risk associated with each allele. For each SNP a p value was calculated which is a measure of the confidence in the odds ratio.

Particular evidence for genetic association to COPD was found for the SNP located at position 184 in SEQ ID NO: 1. This SNP was associated with COPD by both genotype—(p=0.0185) and allele-wise (p=0.0043) tests for association. The fact that both tests are positive for association most likely indicates a multiplicative model of inheritance where the risk for disease increases with each copy of the disease allele. As indicated in Table 1, this SNP is known by the NCBI identifier rs10939644 and is located in the 5′ flanking region of the gene. The evidence for genetic association of this SNP to COPD is shown in Table 2.

TABLE 2
Evidence for the genetic association of SNP rs10939644 (corresponding
to position 184 in SEQ ID NO: 1) to COPD.
Genotype-wise test for association

| | Genotype | | | Summary |
|---|---|---|---|---|
| | GG | GA | AA | statistics |
| Cases | 473 | 188 | 25 | X² = 7.98 |
| Controls | 337 | 98 | 8 | p = 0.0185 |

Allele-wise test for association

| | Freq. allele G | Freq. allele A | |
|---|---|---|---|
| Cases | 82.7% | 17.3% | ODDS = 1.43 |
| Controls | 87.1% | 12.9% | p = 0.0043 |

The allele-wise test for association shows that the “A” allele of SNP rs10939644 is increased in COPD cases compared with controls. This indicates that inheritance of one or more chromosomes bearing the A allele of this SNP increases risk for COPD, with risk being greatest for AA homozygotes, intermediate for heterozygotes and least for GG homozygotes.

The location of a G/A SNP at position 184 in SEQ ID NO: 1 affects the composition of putative transcription factor binding sites in this segment of DNA. While the SNP is located approximately 7 kb from the start of exon 1, sequence elements in the region of the SNP may still regulate FGF-BP1 expression if they behave as enhancer-type elements. The presence of enhancers often comprise short sequence motifs, each enhancer having a consensus sequence, and may regulate gene expression from several kilobases away. Among the putative enhancer binding sites at the position of the indicated SNP, there is a putative T cell-specific enhancer element (consensus sequence MAMAG, where M=A or C). TCF1 is of interest here since this factor regulates T cell differentiation and function, and COPD has a clear inflammatory component. On chromosomes bearing the common G allele, a consensus sequence for TCF1 is found at nucleotides 180-184 in SEQ ID NO: 1. Substitution of the G allele for A at position 184, disrupts this putative TCF1 site, but another is created at nucleotides 181-185. In addition, on chromosomes bearing an A allele, a putative binding site for nuclear factors of activated T cells (NFAT) is created which is absent in the presence of the alternate allele. NFAT is a multimeric transcription factor complex, important in regulating a variety of cell types, including immune cells, and has the consensus binding sequence [T/A]GGAAAA[A/T]. Thus, the presence of one or more putative binding sites for transcription factors at position 184 provides a possible biological rationale for the genetic association.

1. A method for assessing the predisposition and/or susceptibility of an individual to COPD, which method comprises
(a) determining the identity of one or more nucleotides at a polymorphic site in an FGF-BP-1 gene of the individual; and
(b) determining the status of the individual by reference to said one or more nucleotides.

2. A method for assessing the predisposition and/or susceptibility of an individual to COPD, which method comprises:
(a) providing a nucleic acid sample that has been removed from the individual;
(b) determining the identity of one or more nucleotides at a polymorphic site in an FGF-BP-1 gene of the individual; and
(c) determining the status of the individual by reference to said one or more nucleotides.

3. A method according to claim 1 wherein the polymorphic site is a single nucleotide polymorphism site.

4. A method according to claim 1, wherein the method comprises determining the nucleotide of the individual at position 184 of SEQ ID NO: 1.

5. A method according to claim 1, wherein the method comprises determining for either (i) the presence of G at position 184 of SEQ ID NO:1, or (ii) the presence of A at position 184 of SEQ ID NO: 1.

6. A method of treating an individual having, or at risk of having, COPD, with a drug capable of modulating FGF-BP1, which method comprises:
(a) determining the identity of one or more nucleotides at a polymorphic site in an FGF-BP-1 gene of the individual;
(b) determining the status of the individual by reference to said one or more nucleotides; and
(c) administering an effective amount of the drug.

7. A method according to claim 6, wherein the individual is determined as having base A at position 184 of SEQ ID NO:1.

8. Use of a compound capable of modulating FGF-B1 expression or functional activity in the preparation of a medicament for treating COPD.

9. Use of a compound capable modulating FGF-BP1 expression or functional activity in the preparation of a medicament for treating an individual for COPD, wherein the individual has been identified as having a polymorphic nucleotide or a polymorphic nucleotide sequence which is associated with COPD in the FGF-BP1 gene.

10. Use of a drug capable of treating COPD in the preparation of a medicament medicament for treating an individual for COPD, wherein the individual has been identified as having a polymorphic nucleotide or a polymorphic nucleotide sequence which is associated with COPD in the FGF-BP1 gene.

11. Use according to claim 10, wherein the drug is selected from the group consisting of a beta-agonist, an anticholinergic, theophylline, N-acetylcysteine, a combination of a long-acting beta-agonist and an inhaled corticosteroid, and a combination of an anticholinergic and albuterol.

12. Use according to claim 8, wherein the individual is determined as having base A at position 184 of SEQ ID NO:1.

13. A method of screening for a compound which can be used to treat COPD, which method comprises:
(a) providing a sample containing FGF-BP1 polypeptide or a homologue thereof or a fragment of either, and a candidate compound; and
(b) detecting the binding of the FGF-BP1 polypeptide, homologue or fragment, to the candidate compound in the sample.

14. Use of a compound detected by the method of claim 13 in the preparation of a medicament for treating COPD.


SEQUENCE LISTING (2 sequences)

SEQ ID NO: 1 - DNA, 10605 bp, Homo sapiens
  misc_feature (184)..(184): n is G or A
ttgggaaagatatggccttccaaatggacatggcccttactcagaagtttatcacaaaaataacaccaccccagacaacatcacctcttaatttgatgtcaaaatgtcggagtgatgaggtatcaccactacagcaatcatcaaagtgtgagccatagactcacctggtcaggtctaggaaaangagccaaaccatgttcatcttatcttcccttgagtgcctgatacttcaccttgctgagagtggttactgaataatgactgaagagataacatcccctttcccccactgttgagctgtgtttgcataattgcaggctcttgagaaattcttgtctgcctctggtaggctagatgtcccaactcaaagcaccacagctttccctccaacatagtcattaacctctgtgtggtcatgactgatatccttgacgtttcccctattgtctgctaactcctcaagggtaaggaccatttttcttgactgcacttccaggcaatcattaacactgagggcctggacatagtaggtgcttcatgcgtgcttttaggatgaatgaaaaaatgactgagtggtagtagaaatgaggtcgcattagggtccagccttgcagagtctcattcccctgctcattcttgtcttctcctaagcagcatgccacatgtgtgagtccttttgctctttgtgtaagggcattcagcacagaagggtatctttctgtgaggtgaggtcctccttgaacactccatcacgggttgacattcactgttgggcccctgagcattaggaccctctgaagatactcacccatttgattccaaatttggaatcaaatttggtatccttcagcgctgaatgcccttacaggtcttttcagaggtgcttttctggtttgatttttctaaatattgtcatctcttcatgtgaagaaagggatgtggtcataagccaatgtgcctcgtgactctcagccttaacaccaagaaagggcagcttaccttctccctttttcagctttcaaagggagcaactgttgctctttgtagcacacatgggctagaggctgaaaatccaaaagggagactctctaggtccttctttttctccaagataggaagggaggtagaggtggcatacagaaatgtgtacttgtgtggggttttgtgatgtgtgagcatgtgtgtgggaatgggtgtgtatatgtgagtgctttcttaagtgcatgtatgtgggggtaccttgaaaaatgttgtccaaaagacacagacctctttaaagggtgtttgactgtttaagaatgatttacttaaaaacaaagatccaaaaagcaaagaagtgatcaaaaaacttgatttgtaggggcaattgattatagaagagctaaaaaaaaaaaacttttacaaaaagaaaacactgtaaaaggctttgaaatagccctattacttgagaaaaaatacctattattattaagtggtttcattcaaatggctgatattctagcatttttgacatactacaaatagtagtttcgtatgatttaacctaacacaaaaattggagttaggtttcaaatgtgttgagaatcagaaccaaccattaaacattgtttctaaaaacatgttgatatgattccactgctgggtatatacccaaatgaaaatcattctacaaaaaagtaaataaataaaaatttaaaaaaacaatatgcactcatatgttcattgtggtaatattcacaacagcaaaaacatggaatcaacctggatgcccatcaaaggtgaattgacaaaagaaaatgtggtacatatacattatgggatactatctatgcagccacgaaaaataatgaattgtgtcctttgcagtaacatggatgcagctggaggctattatcctaaacaaattaatacaggaatagaaaaccaaatactgcatgttctcacttataagagaaggagctagaccgagcacagtggctcacgactataatcccaacactttgggaggccgaggcaggcggatcatgaggtccggagttcgagagcagcctggccaccatggtgaaaccccgtgtctactaaaaataaaaaaattagccaggcatggtggcaggtgcctgtaatcccagctgctcggaaggctgaggcaggagaactgcttgaagccagaaggtggaggttgcagtgagccaagatcgtgccactgcactctagcctgggtgacagagcaagactttgtctcggaaagaaaaaaaaaagagaaggagctaaatattaggtaagcatggacataaagatggcaaaaatagggctggagactactagagtggggagaaatggaggggggcaaggttagaaaaactacctactgggtactatgctcactacctgagtgataagatcatttgtacagcaaacctcagcgacatgcaatttacccatgtaacaaatctacacatgtaccctctgaacctaaaataaaagttaagaaatgtgttaatattatgtggttaataacaacattctttctaaagtaaataaacttaataggttttcaccttgcaatcatacccaatactatagaatccacactctgctatctcttcattttctaaagaaataaaactttcattgtcctgaacaggaatcctgaaatctcatgatgaatgagcactttaaatttggaatgcaaatattctttttttttttgtctcactttgtcgcccaggctggagtgcagtggcacatctcggctcactgcaagctccgcctcctgggttcacgccattctcctgcctcggcctcctgagcagctgggactacaggcacccgccaccatgcctagctaatttttgtgtgtgtgtgtgtgtttttagtagagacagggtttcaccatgttagccaggatggtctcgatctcctgacctcatgatccgcctgccctggcctcccagaatgcaaatattcttgagtaggtctaagattaatgctttttgccagatgaattagattttactgatgtttgcttagcccaatttccttattttctgataataaatgtattttaatgcacattatttgcccaacaaattaatattgctttagtgcagcagctcagcatttgcaatgtctgtttgtaaagtaacgtttgaaggaagggttgatttttgacaactttggcctggcaataagtttggcacagttaagacttacctcagaaccaaattgcacccattatatgaataatctctggtgactgcataagtagggccaggcctctaggtcaagaagctatgtggtagtaacaggcatgagagtaaaatcagcttttgaacttcaaatccccagctggtgaccagacatgcattcaattaccagccttagccagtgtgtttttaccaattgtgttcagacttcctttcacaatcccaaagaaagtattcatcatttaagttttgtgtgatcatgagaagaataacatgatcccaatctaaccagaacatgccagacaatttccaaattaaggacattctcaaaacacctaaccaattcttctgcaaactgccatagtcatcaaaaacaagcaacatctgagaaacagccatagccaagaagaacccaaaaagtcacgatcactaaatgcaacctggtgtcatgttgggatcctggacagaaaaagaacattaggagctgaaaatttaattctacaccaaacctgcacatgcatgtttatagcagctctattcatcactgtcaaaatttagaggcatccaaaatgtcctagatgaatgaacaaatgaactcgggtacatccaatcaatggaattttagtagtaggtgaatgaataaagggacttggatatatccaatctttggaattttatcagcacttaagagaaatgtgctatcaagttatgaaaaacatagagaaaccttaagtgcatattactaggtaaaaaagtcaatataaaaggttacatattgtgtgattccaactatttgaaattcttaaaaagataaaactgaggagacagcaaaaggatgagcattgcctggggttgtaggggatggagtggtaagtaggcagaacacagagaatttttagggcagctaaactattctgtatgataccatagtggtgggtaccgtcattatacctttcttagttaattgatattatgcatgtcattatacatttctcaaaacccaaagaatgtacagcacttagagtgaacctaatggaaatgatggacattgggtgataatgacatgttaatgtaggtttattacttacagcaaatgtactaccctggttcatgatgttgaaggtaggggagactgtgcctgcgtgtggggaggggtatatgggacttttgatactttccactcaattgtgctatgagcataaaactactctaaaaaataaagtctgtcaataataataataataataataaaaactagaacaacaggtaaatcctaagcaagtctgaataaagtatggacttcaattgacaataatgtatcaatgtttgtcagttgtgacaaacgtaccatagtaatgtaacaggtaaatagggaacccaggtgagaattggaatgataactatctttgcaattctctgtaaacctaaaattattctaaaactttaaaaaaaaatttaaagggaaggtatgaaggggcaaagagaggagaattccacaatttatcagtgtatgcgtacactgcctcatttgtgtttagaatgatagcagcggcctccagataattcttctctaattgtgtgtcagacactgtgcttagtccccctaagccttgcctccacatctgtcccatgaggacctgggccgactttcctcctggggttcttgtgcaatgggcctttatgtactcatccccaagaattacagagcccctcagtgctgggagcatggggaggtggaggggcctgaagttactggaacctgctatgcactgcgtggctggggctccatgtcctctgcagtctccccagagccactggaaaaggacatcgtttccttagttcatggagatacgaagaaagcttgggaaacagccttagcacagtgcctggcacgctgtaagctcattggaggaagtgtgccatttctccccgtggacattcttaaagatgtgggtggcatgcagggcgtcaggagccaatgacaccaacagagaggggctcgcattggagcctggccttgaaggatgaggcggtgtgtgccaggcagagaaggaagggaaggggatcccaagtgcagggaacagcagaggcaaaggcaggaagtgggcgcatgcatggagtatctgcaaatggtgggacagggtttcagtgtggccagattgcagtgtgtgtgtgcatgtgtgtgtgtgtctgtctctgtgtgtgtgtcagtgtcactgtgtgtgattctgtgtgtgtgtgcctatgagtatgtgtgtgaatgtgtctaggtgtcagtatgtgtgtgtctctgtgtgtctatgagtgtggtgtctttgagtgtgtttgtgtgtgtttgagtgtgtcagtgtgtgtttgtgtgtgtctttgtatgtgtgtcagtgtgtatgtgtatttatgtgtctgcgtgtgagtacctggaaatgaggttggaagggcaggatgagccaaggaggtgactgccccacagcctgctctgatgctgtgctgctgagtctagaagggaaagagcaaagcagacgctgggatgggagtcacaggaaggggaagggccacaaggagccagtggggaatgtgcagtgttacctggcaaagctcagagaagcacttcccagaaagaccccggagtccacatgtttccttgtaaatatgttttgtgttccaactgcaatctgcctccagaaagaatctgcctgcctcccaaggatcaagtagaattttccattttgtaggagactccccttgttgagcaaagcagctttgccttgcctatgtccaaataatcctccaaaataccgcatgagagagcctgttcgtggaggtcagggcctcagcggggtgtttgtgaggatagtgaggacggacccctgggtgaggcaattttaggagagaggcacacagcttcagaaatggcttcttccaacagattcatgcagagactgagcctctgagtaaattggtcaacatctatagctatgaccacggaattctcaaacaggtggatcgttcataaaatccaaacacagttacaagttttggcctgcatagtgttgagctacagctttatttttagaaaattttaattagctgccaaccaatttcatataaacacatctggatttggggatgtcattttaaaaatcagacagtgtgacaatactgagcctaatccctgcattttctcagtcaattggagatgagtgttgacctctatggaggcagagactctctagtttgccacagtccccatcactccctattgccttatctcaaacccaaaagtcagttgccatttaatatcacactaaacaatagttttcttatggctgaattaaggaaaagtgaacaatttcttcttctgaaaatctatgaaaggtgaggtgataatatataatgagggccacatcttccctgtagaagtgagtttctttttaaatgctgcatatgtaaataacaaatgtctgttttcaaagtatacaacttaagaccctgtcatgaggcaaaacgattctgctccactttatgttaacagcttagcccagtaaaaatttagtatgcagctataaaggagacgttgttattgtcagcacactgcaaatggatgatgggagaggctaagtggtttgggcatagcactcagctaaacacatgctgtggccaggattccacctgggtcattctggtgtgggactgtgcctgtagcaggcagggcagcctgagctctctgaatgagacctgaaggtaagtatttacaacagtaatcttatattaaaaattccctctttaccagcctctcagagcctggtagaattccctgataaaaaagagggaatttttaatgtaaatttccctgcctctagaattagggtatcctctgcttatgtagtaacttcagatggaaaaaggaactcagacactacgaaaacttcggcaacttcagtcacccattcaacaaagatttattgagagtggcaggcaggaagagatcagaccatggatgtagagaatctcacttttaattttgagatgttccttttcctgcctctgtgaacttggacaagctcttacatatctccagaaccaattcctgcttttgtaaaaggggaggaatcattgcactccctactagagcggatgtgatgagggaaaaggagaactcaggtaaaatgggctcagtagtttggcagctcggatcatgtcattatagttattttactcctccatagacatcactgctggggtcctctccccatcaaggagggtcactgtgaggctgagttgtcctcctatcctggccacagggtggacagggaagggcctagaggaagcgtttgatggcatgcaggtaaccccccttttcactggattacatccagcactttccctgcaggaaccggctccctcggaggggcgtggctgggaggagctgtgagtaacgtgccacagtgttgtaaaaacccagtgagtgttataaaaacccagtcagcctggctcctgttgaatagtctaccccccttgcactctacctgacacagctgcagcctgcaattcactcgcactgcctgggattgcactggatccgtgtgctcagaacaaggtaagagcctcgctcctttaacagctttattacctgctgccacttctcactccaccaagggcctggagttccttgggcctgcattccatcatgggcaggtgggggctgagccccagggccgtgagaaatgactgggtgtttttctggcatatccagtctcctcttcaccatttattgctgttctctgcccttttgtgattgtctttctccttcttaaccctagaagatggccaacttctagggtagaaatcagatcaggaccatttcctgggtctgcctctttttataaaaaattctgtgttttgttttaaatggagcacaaataatacaaatcatcttatcattatgtgttatatgaaactaaattgtcattgttcatagttgtttttcttattctttggcaatgtttttaaagggcagagtaaatgtgaaaagcaaaaagtgatcaaccaggggtgacagaaaggaataaggagattgatctggctacccataccagctttgaacttgcccactatgagatcctgagcaatttacttacctgtctgagccttaagagtcagagctgtcacctgggatgacatattaatagctacctggaagagctcttgtgtggatttcacaaggtgggaatcaaaccctagaccagtacttagcacacaggaggtgtcatatagaaactgacttctcttcttctgttaagcatgggtttttattgtacacatgatcagattcatgcagttcagtgtgacgttggtgttcatcctattgattcaatgatttctcatgatccttgtcttcgatcttgtagtgtgaactatctggttgaaattcacctctctccaccctgattcactttgcccaggcaaatcagacaaccaccccacctcacacctttccttaaactcttacttccccacaaaaagctcccccacccccacaaccccacccaccccacccaaaccccaggcatgttcaactctgggtcaacatcacctttcatgatgcttaccacatcctttctcattttgggcatctgtcatgtcttttctcattttgggcatctgtgtccatatggagtcatgagatagaaatccagccctttggagctgaaagtttggagcagtaacttgtttcatatcagaaacagagatgacgggatagattccctgatcaatgctatttaaattctcccaaagagtggtctctaagctctttcaaatctgaggctttctcaatcatggtgatttcttcaacacctagtctggtggggggcatatagtaggtgctcagtaaatgcttgttgaatgagtgaatgaaagagtgaggagtgagcagatgcaggagttaatgagtgaataaatgataaatgagagttcgtgaggctccaaagagtggtacttttgtgacactcaagactcaagtcaagtggaaatgtgagtagacaggctgtttgtctagtgactccaggagccagaagcgttcttggtctactctgaaatcatgtaggcacctgtcagggtagggggaggccagcgtgtagtcctgcacagctgaactgctgcctgccactgactcacttgccttttgtcaaacaggtgaacgcccagctgcagccatgaagatctgtagcctcaccctgctctccttcctcctactggctgctcaggtgctcctggtggaggggaaaaaaaaagtgaagaatggacttcacagcaaagtggtctcagaacaaaaggacactctgggcaacacccagattaagcagaaaagcaggcccgggaacaaaggcaagtttgtcaccaaagaccaagccaactgcagatgggctgctactgagcaggaggagggcatctctctcaaggttgagtgcactcaattggaccatgaattttcctgtgtctttgctggcaatccaacctcatgcctaaagctcaaggatgagagagtctattggaaacaagttgcccggaatctgcgctcacagaaagacatctgtagatattccaagacagctgtgaaaaccagagtgtgcagaaaggattttccagaatccagtcttaagctagtcagctccactctatttgggaacacaaagcccaggaaggagaaaacagagatgtcccccagggagcacatcaaaggcaaagagaccaccccctctagcctagcagtgacccagaccatggccaccaaagctcccgagtgtgtggaggacccagatatggcaaaccagaggaagactgccctggagttctgtggagagacttggagctctctctgcacattcttcctcagcatagtgcaggacacgtcatgctaatgaggtcaaaagagaacgggttcccttaagagatgtcatgtcgtaagtccctctgtatactttaaagctctctacagtccccccaaaatatgaacttttgtgcttagtgagtgcaacgaaatatttaaacaagttttgtattttttgcttttgtgttttggaatttgccttatttttcttggatgcgatgttcagaggctgtttcctgcagcatgtatttccatggcccacacagctatgtgtttgagcagcgaagagtctttgagctgaatgagccagagtgataatttcagtgcaacgaactttctgctgaattaatggtaataaaactctgggtgtttttcagaaatacattcaaacattgcttggttttctctgtgtctggtcccaaataaactcattaagtagtccccatatatcaatggaaaaacaatctaaacaggaacttccagttccccacccttcgcaagttatctctt

SEQ ID NO: 2 - protein, 234 aa, Homo sapiens [sequence omitted]